(19) United States
(12) Patent Application Publication
(10) Pub. No.: US 2011/0294789 A1
(43) Pub. Date: Dec. 1, 2011

(54) COMPOUNDS FOR RHO KINASE INHIBITION AND FOR IMPROVING LEARNING AND MEMORY

(75) Inventors: Karoly Nikolich, Emerald Hills, CA (US); Laszlo Nadasdi, Oakland, CA (US)

(73) Assignee: AMNESTIX, INC.

(21) Appl. No.: 12/991,846

(22) PCT Filed: May 11, 2009
(86) PCT No.: PCT/US09/43464

§ 371 Date: Jun. 1, 2011

Related U.S. Application Data
(60) Provisional application No. 61/052,600, filed on May 12, 2008.

(51) Int. Cl. A61K 31/5513; A61P 25/24; A61P 25/22; A61P 25/00; A61K 31/497; C07D 401/10

(52) U.S. Cl. 514/218; 514/253.05; 540/575; 544/363

(57) ABSTRACT

The present invention provides a compound of Formula (I) and methods for improving memory, inhibiting rho kinase 1 or 2, inhibiting PIM kinase, or inhibiting IRAK1 kinase in a subject by administering a therapeutically effective amount of the compound.

### INFORMATION ON RELATED APPLICATION

This application claims the priority benefit of U.S. Provisional Application No. 61/052,600 filed on May 12, 2008, which is hereby incorporated herein by reference.

### BACKGROUND

Human memory is a polygenic cognitive trait. Heritability estimates of ˜50% suggest that naturally occurring genetic variability has an important impact on this fundamental brain function. Recent candidate gene association studies have identified some genetic variations with significant impact on human memory capacity. However, the success of these studies depends upon preexisting information, which limits their potential to identify unrecognized genes and molecular pathways.

Recent advances in the development of high-density genotyping platforms have enabled the identification of some of the genes, particularly the KIBRA gene, responsible for episodic and long-term memory performance (Papassotiropoulos et al. Science 2006, 314, 475; WO 2007/120955). However, there is still no treatment available for subjects suffering from deteriorating episodic or long-term memory. Based on the identification of KIBRA as a central protein within the signaling pathway for stimulation of memory, it was found that administration of rho kinase 2 (ROCK) inhibitors, particularly Fasudil, can enhance learning and memory (Huentelman et al. Behavioral Neuroscience 2009, 123, 218; WO 2008/019395). In order to realize a treatment suitable for subjects suffering from deteriorating episodic or long-term memory, new compounds preferably with improved and/or more selective inhibitory effect on ROCK are needed. Such compounds are suitable for the enhancement of learning and memory.

### SUMMARY

In one aspect, compounds of the following Formula I are provided:

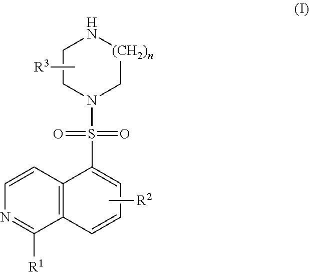

wherein R¹ is a member selected from the group consisting of hydrogen, C₁₋₆ alkyl, hydroxy, and halogen, preferably from the group consisting of hydrogen and C₁₋₆ alkyl; R² is a member selected from the group consisting of C₁₋₆ alkyl, halogen, —C(O)—R⁴, C₁₋₆ alkoxy, C₁₋₆ haloalkyl, —C(O)N(R⁴)R⁴, —N(R⁴)—C(O)—R⁴, —N(R⁴)R⁴, and —C(O)OR⁴, whereas R² is localized at position 6, 7, or 8, preferably at position 8 of the isoquionline moiety; R³ is a member selected from the group consisting of hydrogen, and C₁₋₆ alkyl; each R⁴ is independently a member selected from the group consisting of hydrogen, C₁₋₆ alkyl and C₃₋₈ cycloalkyl; and n is 0, 1, or 2, preferably 1 or 2; and salts, hydrates and solvates thereof.

Additionally, methods are provided for inhibiting ROCK using a compound of Formula I. Thus, the compounds of Formula I can be used for treating subjects with ROCK related conditions and diseases, e.g. vasospasms following subarachnoid hemorrhage, angina pectoris (e.g. Prinzmetal's or vasospastic angina), conditions following spinal cord injury or injuries of the brain (such as stroke, traumatic brain injury), heart failure-associated diseases (e.g. due to vascular resistance and constriction), myocardial infarction, pulmonary arterial hypertension essential hypertension, atherosclerosis and aortic stiffness, and peripheral vascular diseases like Reynaud's phenomenon, and erectile dysfunctions which are in need of ROCK inhibition.

Further, methods are provided for improving learning and memory (including improving cognitive deficits in psychiatric disease such as schizophrenia, treating dementia, such as Alzheimer's disease, Pick's disease, Fronto-temporal dementia, vascular dementia, Kuru, Creutzfeld-Jakob disease, and dementia caused by AIDS/HIV infection), improving neural plasticity, amnestic subtype-mild cognitive impairment, age-associated memory impairment, and/or treating Alzheimer's disease in a subject, the method comprising administering to a patient in need thereof, a therapeutically effective amount of a compound of Formula I.

In other aspects, methods are provided for improving memory or treating rho kinase 1 and/or 2 related conditions by administering to a patient in need thereof a therapeutically effective amount of a compound of Formula 1.

In another aspect, methods are provided for treating PIM kinase related conditions in a subject, the method comprising administering to a patient in need thereof, a therapeutically effective amount of a compound of Formula 1. In some embodiments, the condition is selected from the group consisting of ALL, CLL, AML, or CML, Hodgkin-Lymphoma and Non-Hodgkin Lymphoma.

In another aspect, methods are provided for treating IRAK1 kinase related conditions in a subject, the method comprising administering to a patient in need thereof, a therapeutically effective amount of a compound according to Formula 1. In some embodiments, the condition is selected from the group consisting infection, atherosclerosis, sepsis, auto-immune diseases and cancer.

Other objects, features and advantages will become apparent from the following detailed description. The detailed description and specific examples are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE DRAWINGS

FIG. 1: Scheme for the synthesis of 1-(8-methyl-5 isoquinoline-sulfonyl)homopiperazine. The first step represents the creation of the isoquinoline moiety by adding aminoacetylaldehyde dimethyl acetal (H₂NCH₂CH(OCH₃)₂), ethyl chloroformate (ClCO₂Et), trimethy phosphate (P(OMe)₃), and titanium tetrachloride (TiCl₄). The next step is a sulphonylation with sulphuric acid and oleum (SO₃/H₂SO₄). The last step is the addition of the homopiperacive moiety by adding thionyl chloride and homopiperacine (SOCl₂/homopiperacine).

FIG. 2: Scheme for the synthesis of 1-(1-chloro-8-methoxy-5 isoquinoline-sulfonyl)homopiperazine. The first step represents the creation of the isoquinoline moiety by adding aminoacetylaldehyde dimethyl acetal (H₂NCH₂CH(OCH₃)₂), ethyl chloroformate (ClCO₂Et), trimethy phosphate (P(OMe)₃), and titanium tetrachloride (TiCl₄). The next step is the creation of an N-oxide with hydrogen peroxide and acetic acid (H₂O₂/AcOH). The next step is the introduction of the chloride residue with phosphoryl chloride (POCl₃). The next step is a sulphonylation with sulphuric acid and oleum (SO₃/H₂SO₄). The last step is the addition of the homopiperacive moiety by adding thionyl chloride and homopiperacine (SOCl₂/homopiperacine).

FIG. 3: Scheme for the synthesis of 1-(1-hydroxy-8-acetyl-5 isoquinoline-sulfonyl)homopiperazine and 1-(8-acetyl-5 isoquinoline-sulfonyl)homopiperazine. The first step is the generation of a hydroxyethyl residue by adding n-butyl lithium (BuLi) and acetaldehyde (CH₃CHO). Next step is an oxidation with sodium dichromate (Na₂Cr₂O₇). The next step is a sulphonylation with sulphuric acid and oleum (SO₃/H₂SO₄). The next step is the addition of a fmoc protected homopiperacive moiety by adding thionyl chloride and finoc-homopiperacine (SOCl₂/fmoc-homopiperacine). The next step is the creation of an N-oxide with hydrogen peroxide and acetic acid (H₂O₂/AcOH). The last step is the hydroxylation and cleavage of the fmoc-group with acetanhydride and sodium hydroxide (Ac₂O/NaOH).

FIG. 4: Scheme for the synthesis of 1-(1-hydroxy-7-acetyl-5 isoquinoline-sulfonyl)homopiperazine and 1-(7-acetyl-5 isoquinoline-sulfonyl)homopiperazine. The first step represents the creation of the isoquinoline moiety by adding aminoacetylaldehyde dimethyl acetal (H₂NCH₂CH(OCH₃)₂), ethyl chloroformate (ClCO₂Et), trimethy phosphate (P(OMe)₃), and titanium tetrachloride (TiCl₄). The next step is the generation of a hydroxyethyl residue by adding n-butyl lithium (BuLi) and acetaldehyde (CH₃CHO). Next step is an oxidation with sodium dichromate (Na₂Cr₂O₇). The next step is a sulphonylation with sulphuric acid and oleum (SO₃/H₂SO₄). The next step is the addition of a fmoc protected homopiperacive moiety by adding thionyl chloride and fmoc-homopiperacine (SOCl₂/fmoc-homopiperacine). The next step is the creation of an N-oxide with hydrogen peroxide and acetic acid (H₂O₂/AcOH). The last step is the hydroxylation and cleavage of the fmoc-group with acetanhydride and sodium hydroxide (Ac₂O/NaOH).

FIG. 5: Scheme for the synthesis of 1-(1-methyl-8-carboxamide-5 isoquinoline-sulfonyl)homopiperazine and 1-(1-ethyl-8-carboxamide-5 isoquinoline-sulfonyl)homopiperazine. The first step is the alkylation with dibenzoylperoxide and alkyliodide ((PhCOO)₂/Alkyliodid). The next step is a carboxylation with n-butyl lithium (BuLi) and corbonoxide (CO₂). The next step is the generation not the carboxamide by adding thionyl chloride in methanol (SOCl₂/MeOH) and ammonia in methanol (NH₃). The next step is a sulphonylation with sulphuric acid and oleum (SO₃/H₂SO₄). The last step is the addition of the homopiperacive moiety by adding thionyl chloride and homopiperacine (SOCl₂/homopiperacine).

FIG. 6: Scheme for the synthesis of 1-(1-methyl-7-carboxamide-5 isoquinoline-sulfonyl)homopiperazine and 1-(1-ethyl-7-carboxamide-5 isoquinoline-sulfonyl)homopiperazine. The first step represents the creation of the isoquinoline moiety by adding aminoacetylaldehyde dimethyl acetal (H₂NCH₂CH(OCH₃)₂), ethyl chloroformate (ClCO₂Et), trimethy phosphate (P(OMe)₃), and titanium tetrachloride (TiCl₄). The next step is the alkylation with dibenzoylperoxide and alkyliodide ((PhCOO)₂/Alkyliodid). The next step is a carboxylation with n-butyl lithium (BuLi) and corbonoxide (CO₂). The next step is the generation not the carboxamide by adding thionyl chloride in methanol (SOCl₂/MeOH) and ammonia in methanol (NH₃). The next step is a sulphonylation with sulphuric acid and oleum (SO₃/H₂SO₄). The last step is the addition of the homopiperacive moiety by adding thionyl chloride and homopiperacine (SOCl₂/homopiperacine).

FIG. 7: Scheme for the synthesis of 1-(8-aminoacetyl-5 isoquinoline-sulfonyl)homopiperazine. The first step is the introduction of a thiocyante group by adding potassium thiocyanate (KSCN) and brome (Br₂). The next step is a saponification with hydrochloric acid (HCl(aq)) and ethanol (EtOH). The next step is an oxidation with potassium permanganate (KMnO₄). The next step is an acetylation with acetanhydrite (Ac₂O). The last step is the coupling of the homopiperazine moiety by adding thionyl chloride and homopiperacine (SOCl₂/homopiperacine).

FIG. 8: Scheme for the synthesis of 1-(6-aminoacetyl-5 isoquinoline-sulfonyl)homopiperazine. The first step is a sulphonylation with sulphuric acid and oleum (SO₃/H₂SO₄). The next step is an acetylation with acetanhydrite (Ac₂O). The next step is the addition of a Nboc protected homopiperacive moiety by adding thionyl chloride and Nboc-homopiperacine (SOCl₂/Nboc-homopiperacine). The last step is the cleavage of the boc-group hydrochloric acid and iso-propanole (HCl/i-PrOH).

FIG. 9: Scheme for the synthesis of 1-(7-aminoacetyl-5 isoquinoline-sulfonyl)homopiperazine. The first step represents the creation of the isoquinoline moiety by adding aminoacetylaldehyde dimethyl acetal (H₂NCH₂CH(OCH₃)₂), ethyl chloroformate (ClCO₂Et), trimethy phosphate (P(OMe)₃), and titanium tetrachloride (TiCl₄). The next step is a sulphonylation with sulphuric acid and oleum (SO₃/H₂SO₄). The next step is the addition of a Nboc protected homopiperacive moiety by adding thionyl chloride and Nboc-homopiperacine (SOCl₂/Nboc-homopiperacine). The next step is the coupling of an amino group with copper, copper(I)bromide, and ammonia (Cu/Cu(I)Br/NH₃). The next step is an acetylation with acetanhydrite (Ac₂O). The last step is the cleavage of the boc-group hydrochloric acid and iso-propanole (HCl/i-PrOH).

FIG. 10: Scheme for the synthesis of 1-(8-aminomethyl-5 isoquinoline-sulfonyl) 2-methyl-piperazine. The first step is a sulphonylation with sulphuric acid and oleum (SO₃/H₂SO₄). The next step is the addition of a Nboc protected homopiperacive moiety by adding thionyl chloride and Nboc-homopiperacine (SOCl₂/Nboc-homopiperacine). The next step is the coupling of the amino group with tris(dibenzylideneacetone)dipalladium (Pd(dba)₂), 2,2′-bis(diphenylphosphino)-1,1′-binaphthyl (BINAP) and methylamine (MeNH₂). The last step is the cleavage of the boc-group hydrochloric acid and iso-propanole (HCl/i-PrOH).

FIG. 11: Scheme for the synthesis of 1-(1-methyl-8-trifluoromethyl-5 isoquinoline-sulfonyl) 2-methyl-piperazine. The first step represents the creation of the isoquinoline moiety by adding aminoacetylaldehyde dimethyl acetal (H₂NCH₂CH(OCH₃)₂), ethyl chloroformate (ClCO₂Et), trimethy phosphate (P(OMe)₃), and titanium tetrachloride (TiCl₄). The next step is the synthesis of the Reissert compound using benzoylchloride (PhCOCl) and trimethylsilylcyanide (TMS-CN). The next step is the methylation with sodium hydride, methyl iodide, and sodium hydroxid (NaH, MeI, NaOH). The next step is a sulphonylation with sulphuric acid and oleum (SO₃/H₂SO₄). The next step is the addition of a Nboc protected homopiperacive moiety by adding thionyl chloride and Nboc-homopiperacine (SOCl₂/Nboc-homopiperacine). The last step is the cleavage of the boc-group hydrochloric acid and iso-propanole (HCl/i-PrOH).

FIG. 12: Graphic representation of ROCK-enzyme-inhibition. The individual bars show the amount of remaining rock-enzyme activity (y-axis) after incubation with test compounds (x-axis) in increasing final concentration (0.1 to 100 μM). White bar: vehicle (sham); black bars: Fasudil; dark grey: 1-(8-methyl-5 isoquinoline-sulfonyl)homopiperazine, light grey bars: 1-(1-chloro-8-methoxy-5 isoquinoline-sulfonyl)homopiperazine.

FIG. 13A: Interactions between selected kinases and 1-(8-methyl-5 isoquinoline-sulfonyl)homopiperazine (“methyl-fasudil”) as well as 1-(1-chloro-8-methoxy-5 isoquinoline-sulfonyl)homopiperazine (“methoxy-fasudil”) in comparison to Fasudil which served as a control, measured in an AMBIT KinomeScan. Kinases with more than 50% binding affinity to that compounds at 10 μM concentration are marked with a black box. Kinases with 50% or less binding affinity to that compounds at 10 μM concentration are marked with a grey box. Only kinases are compiled in this table which exhibit binding affinity of more than 50% to either Fasudil, 1-(8-methyl-5 isoquinoline-sulfonyl)homopiperazine or 1-(1-chloro-8-methoxy-5 isoquinoline-sulfonyl)homopiperazine.

FIG. 13B: List of Kinases that have less than 50% binding affinity to either to either Fasudil, 1-(8-methyl-5 isoquinoline-sulfonyl)homopiperazine or 1-(1-chloro-8-methoxy-5 isoquinoline-sulfonyl)homopiperazine measured in an AMBIT KinomeScan.

FIG. 14: Affinities of Fasudil, 1-(8-methyl-5 isoquinoline-sulfonyl)homopiperazine and 1-(1-chloro-8-methoxy-5 isoquinoline-sulfonyl)homopiperazine exemplarily for kinases of the AGC-family. ROCK1 and 2 as well as PKA belong to this family. The hierarchical clustering represents the relationship between that kinases. Binding affinities of either of the two test compounds of more than 50% compared to control are depicted in black, less than 50% in grey.

FIG. 15: Bar graph representation of neurite outgrowth assay. Changes in neurite lengths are shown as % of control (sham, solvent). Two different concentrations of test-compounds (1.5 and 15 μM) were tested on primary hippocampal neurons with Fasudil which served as a control and with the test compound 1-(8-methyl-5 isoquinoline-sulfonyl)homopiperazine (“methyl fasudil”) and 1-(1-chloro-8-methoxy-5 isoquinoline-sulfonyl)homopiperazine (“methoxy fasudil”). The error bars indicate SEM.

FIG. 16A: Induction of LTP by theta burst stimulation. Slopes (30 to 70% of maximum fEPSP amplitude) are plotted vs. time. LTP was induced after 15 min of control recording (arrow). The bars above data points indicate SEM.

FIG. 16B: Effect of 10 μM Fasudil on LTP induction. Mean slopes (30 to 70% of maximum fEPSP amplitude) are plotted vs. time. LTP was induced after 30 min. of control recording (arrow) Black line indicates presence of Fasudil, bars indicate SEM. The hatched line indicates the mean LTP level of the control (130%, see FIG. 16A).

FIG. 16C: Effect of 1 μM 1-(8-methyl-5 isoquinoline-sulfonyl)homopiperazine on LTP induction. Slopes (30 to 70% of maximum fEPSP amplitude) are plotted vs. time. LTP was induced 30 min after onset of Fasudil application (arrow). The black line indicates presence of 1 μM 1-(8-methyl-5 isoquinoline-sulfonyl)homopiperazine, bars above data points indicate SEM. The hatched line indicates the mean LTP level of the control (130%, see FIG. 16A).

FIG. 16D: Effect of 10 μM 1-(8-methyl-5 isoquinoline-sulfonyl)homopiperazine on LTP induction. Slopes (30 to 70% of maximum fEPSP amplitude) are plotted vs. time. LTP was induced 30 min after onset of Fasudil application (arrow). The black line indicates presence of 1 μM 1-(8-methyl-5 isoquinoline-sulfonyl)homopiperazine, bars above data points indicate SEM. The hatched line indicates the mean LTP level of the control (130%, see FIG. 16A).

FIG. 16E: Effect of 100 μM 1-(8-methyl-5 isoquinoline-sulfonyl)homopiperazine on LTP induction. Slopes (30 to 70% of maximum fEPSP amplitude) are plotted vs. time. LTP was induced 30 min after onset of Fasudil application (arrow). The black line indicates presence of 1 μM 1-(8-methyl-5 isoquinoline-sulfonyl)homopiperazine, bars above data points indicate SEM. The hatched line indicates the mean LTP level of the control (130%, see FIG. 16A).

FIG. 16F: Effect of 1 μM 1-(1-chloro-8-methoxy-5 isoquinoline-sulfonyl)homopiperazine on LTP induction. Slopes (30 to 70% of maximum fEPSP amplitude) are plotted vs. time. LTP was induced 30 min after onset of Fasudil application (arrow). The black line indicates presence of 1-(1-chloro-8-methoxy-5 isoquinoline-sulfonyl)homopiperazine, bars above data points indicate SEM. The hatched line indicates the mean LTP level of the control (130%, see FIG. 16A).

FIG. 16G: Effect of 10 μM 1-(1-chloro-8-methoxy-5 isoquinoline-sulfonyl)homopiperazine on LTP induction. Slopes (30 to 70% of maximum fEPSP amplitude) are plotted vs. time. LTP was induced 30 min after onset of Fasudil application (arrow). The black line indicates presence of 1-(1-chloro-8-methoxy-5 isoquinoline-sulfonyl)homopiperazine, bars above data points indicate SEM. The hatched line indicates the mean LTP level of the control (130%, see FIG. 16A).

FIG. 16H: Effect of 100 μM 1-(1-chloro-8-methoxy-5 isoquinoline-sulfonyl)homopiperazine on LTP induction. Slopes (30 to 70% of maximum fEPSP amplitude) are plotted vs. time. LTP was induced 30 min after onset of Fasudil application (arrow). The black line indicates presence of 1-(1-chloro-8-methoxy-5 isoquinoline-sulfonyl) homopiperazine, bars above data points indicate SEM. The hatched line indicates the mean LTP level of the control (130%, see FIG. 16A).

FIG. 17: Graphic representation of LTP data. Mean slopes (30 to 70% of maximum fEPSP amplitude) are plotted for sham control (black; A); 10 μM fasudil (dark grey; B); 1, 10 & 100 μM 1-(8-methyl-5 isoquinoline-sulfonyl)homopiperazine (medium gray; C, D, E); and 1, 10 & 100 μM 1-(1-chloro-8-methoxy-5 isoquinoline-sulfonyl)homopiperazine (light grey; F, G, H). Bars indicate SD.

### DETAILED DESCRIPTION

New compounds are provided that are suitable as ROCK inhibitors, for methods of treating ROCK related conditions and diseases, e.g. vasospasms following subarachnoid hemorrhage, for methods for enhancing memory and learning, for improving neural plasticity, and for treating Alzheimer's disease.

The compounds described herein can be used not only to treat memory loss, which is a symptom of Alzheimer's disease, but can be used to treat a cause of Alzheimer disease and delay onset or prevent development of the disease. Without being held to a particular theory of action, it is thought that the KIBRA gene pathway is related to development of neurofibrillary tangles.

Perhaps the two most studied proteins linked to memory are PKC and cyclic AMP response element binding protein (CREB). PKC family members play a purported role in memory due to their overexpression in several key brain regions, their involvement in memory processes across several species, their age-related alterations in activity in humans correlated with spatial learning deficits, and finally the evidence that PKC inhibition impairs learning and memory (Micheau, J. & Riedel, G. Cell Mol Life Sci 55, 534-48 (1999); Pascale, A., et al. Mol Neurobiol 16, 49-62 (1998); Sun, M. K. & Alkon, D. L. Curr Drug Targets CNS Neurol Disord 4, 541-52 (2005); Birnbaum, S. G. et al. Science 306, 882-4 (2004); Etcheberrigaray, R. et al. Proc Natl Acad Sci USA 101, 11141-6 (2004); Ruiz-Canada, C. et al. Neuron 42, 567-80 (2004)). Support for CREB as a memory-related gene include its defined role in long-term facilitation in the sea slug, Aplysia, and potentiation in rodents, the demonstration that the inducible disruption of CREB function blocks memory in mice, and exploration into compounds that alter CREB activity as memory enhancers (Josselyn, S. A. & Nguyen, P. V. Curr Drug Targets CNS Neurol Disord 4, 481-97 (2005); Carlezon, W. A., et al. Trends Neurosci 28, 436-45 (2005); Cooke, S. F. & Bliss, T. V. Curr Opin Investig Drugs 6, 25-34 (2005); Josselyn, S. A., Kida, S. & Silva, A. J. Neurobiol Learn Mem 82, 159-63 (2004); Martin, K. C. Neurobiol Learn Mem 78, 489-97 (2002); Lonze, B. E. & Ginty, D. D. Neuron 35, 605-23 (2002); Si, K., Lindquist, S. & Kandel, E. R Cell 115, 879-91 (2003); Chen, A. et al. Neuron 39, 655-69 (2003)). Additionally, there is mounting genetic evidence supporting the role of other proteins in memory including HTR2A, BDNF, and PKA (Alonso, M. et al. Learn Mem 12, 504-10 (2005); Bramham, C. R. & Messaoudi, E. Prog Neurobiol 76, 99-125 (2005); Papassotiropoulos, A. et al. Neuroreport 16, 839-42 (2005); de Quervain, D. J. et al. Nat Neurosci 6, 1141-2 (2003); Reynolds, C. A., et al. Neurobiol Aging 27, 150-4 (2006); Arnsten, A. F., et al. Trends Mol Med 11, 121-8 (2005); Quevedo, J. et al. Behav Brain Res 154, 339-43 (2004)).

KIBRA was recently identified in a yeast two hybrid screen as the binding partner for the human isoform of dendrin, a putative modulator of synaptic plasticity (Kremerskothen, J. et al., Biochem. Biophys. Res. Commun. 300, 862 (2003)). A truncated form, which was expressed in the hippocampus, lacks the first 223 aa and contains a C2-like domain, a glutamic acid-rich stretch and a protein kinase C(PKC) ζ-interacting domain (de Quervain, D. J. et al., Nat. Neurosci. 6, 1141 (2003)). PKC-ζ is involved in memory formation and in the consolidation of long-term potentiation (Bookheimer, S. Y. et al., N. Engl. J. Med. 343, 450 (2000); Milner, B. Clin. Neurosurg. 19, 421 (1972)). The C2-like domain of KIBRA is similar to the C2 domain of synaptotagmin, which is believed to function as the main Ca²⁺ sensor in synaptic vesicle exocytosis (Freedman, M. L. et al., Nat. Genet. 36, 388 (2004); Schacter, D. L. & Tulving E. Memory systems (MIT Press, Cambridge, 1994)). The memory-associated KIBRA haplotype block and SNP described in WO 2008/019395 map within the truncated KIBRA, which contains both the C2-like and the PKC-ζ-interacting domains. Taking these findings together, KIBRA seems to play a role in normal human memory performance.

In addition, while KIBRA has high expression in the brain and modulates Ca²⁺ and is a PKC substrate and a synaptic protein, there are several other genetic findings that have allowed the identification of RhoA/ROCK as a target in memory and Fasudil as a modulator to enhance memory, learning and cognition (Huentelman et al. Behavioral Neuroscience 2009, 123, 218; WO 2008/019395). CLSTN2 has high expression in brain, regulates Ca²⁺, and is a synaptic protein. CAMTA1 has high expression in brain, modulates Ca²⁺, and is a transcription factor. SEMA5A has high expression in the developing brain and is involved in axonal guidance. TNR has high expression in the brain, is involved in the ECM, and assists in synapse maintenance. Finally, NELL2 also has high expression in brain, assists in neuronal growth, and shows enhanced LTP but impaired HPF-mediated learning. In addition, in situ hybridization of every one of the genetic targets shows expression in the mouse hippocampus.

The significance of the RhoA/ROCK pathway in normal memory function as well as in Alzheimer's cognitive decline (and likely other amnestic disorders) cannot be overstated. Many devastating disorders include memory loss as a primary clinical characteristic and, in the case of these disorders, the RhoA/ROCK pathway may play a role in their overall severity, progression, or pathology. Even minimal prolongation before memory loss onset would be beneficial to patients suffering from these disorders.

Rho kinase 2 (ROCK) is a serine/threonine-specific protein kinase, which is activated by GTP-bound RhoA. It is a key player of many signaling transduction pathways and controls various cellular functions, including smooth muscle contraction, actin-cytoskeleton remodeling, cell motility and synaptic remodeling. ROCK mediates Rho signaling and reorganizes actin cytoskeleton through phosphorylation of several substrates that contribute to the assembly of actin filaments and contractility. For example, ROCK inactivates myosin phosphatase through the specific phosphorylation of myosin phosphatase target subunit 1 (MYPT1) at Thr696, which results in an increase in the phosphorylated content of the 20-kDa myosin light chain (MLC20). The ROCK inhibitory effect of a test compound can be assayed by incubating the purified kinase and its substrate in the presence of the test compound in comparison to the control without the test compound. The phosphorylated substrate can be detected with specific antibodies and its amount is a measure for the compound's inhibitory effect.

Active-site dependent competition binding assays can be performed with hundreds of known kinases in parallel (Fabian et al., Nat. Biotechnol. 2005, 23, 329; Karaman et al., Nat. Biotechnol. 2008, 26, 127) in order to determine how compounds bind to both intended and unintended kinases. Such methods allow the evaluation of the specificity of a kinase inhibitor. Currently, compounds known as ROCK inhibitors, such as Fasudil, inhibit not only ROCK but also other kinases such as protein kinase A, which plays an important role in cells and living bodies. Accordingly, it is suggested that the inhibition of a protein kinase A activity may cause severe side effects. Therefore, from the viewpoint of using a ROCK inhibitor as a therapeutic agent, it has been desired to develop a compound that can more selectively inhibit ROCK involved in a disease or condition, but not substantially affect activities of other kinases. Therefore, in one embodiment this invention provides compounds which exhibit more specific ROCK inhibition and methods to use those compounds for selectively inhibiting of ROCK.

To measure the effect of the administration of a compound on memory performance in vivo, various known animal tests can be used, e.g. the Sacktor-disc test which is a special form of active place avoidance with the experimental advantages of rapid hippocampus-dependent acquisition and persistent hippocampus-dependent recall (Pastalkova et al., Science 2006, 313, 1141). The apparatus consists of a slowly rotating platform that is open to the room environment. The platform can be energized when the animal runs into a predefined sector. The rotation brings the animal into the shock zone, and the animal rapidly learns to avoid the shock by actively moving to the nonshock areas of the environment.

In another example, the Morris water maze can be used. This in vivo memory test was originally developed to test a rat's ability to learn, remember and to go to a place in space defined only by its position relative to distal extramaze cues (Morris et al., J Neurosci Methods 1984, 11, 47).

Alternatively, one can use a radial arm maze to test animal's memory. It consists of e.g. eight elevated arms around a octagonally shaped central platform. Animals can navigate through the maze using extramaze visual cues as orientation landmarks. Four of the arms are randomly baited with a small food pellet as reward and four are non-baited. Animals are allowed to explore the maze and memorize the locations of baited arms. In follow-up trials, running in a non-baited arm is counted as a reference memory error: re-entry in the same arm is counted as a working memory error as well as re-entry of a previous visited baited arm. Advantageously, the radial arm maze can be used to test working memory as well as spatial memory simultaneously.

Further known behavioral animal tests such as T-maze, open field, or object recognition can be used to assess animal memory. Such in vivo tests can be applied to certain animal subpopulations such as aged animals, disease model animals, etc. in order to particularly assess the memory and memory enhancing effects within such a subpopulation.

A form of classical conditioning is fear conditioning. It belongs to a model for studying emotional learning and memory. Conditioning means pairing of a conditioned stimulus e.g. a light or a tone with an unconditioned stimulus e.g. a mild shock. The unconditioned stimulus alone leads to a fear response. After several trials of repeated pairing the animal shows a fear response also to the conditioned stimulus alone. This is called a conditioned response. Pairing of different stimuli as described above is also known as cued fear conditioning, whereas contextual fear conditioning describes a fear response to the test chamber itself. The cued fear conditioning is sensitive to a brain structure called the amygdala and the occurring contextual response seems to be more sensitive to the hippocampus. In animals both, fear conditioning paradigms as well as active and passive avoidance paradigms, could be used to demonstrate enhanced learning. Such in vivo tests can be used on certain animal subpopulations such as aged animals, disease model animals, etc. in order to particularly assess the memory and memory enhancing effects within such a subpopulation

The effect of long-term potentiation (LTP) can be measured in vitro and is generally thought to correlate with memory performance. Stimulation of an afferent neuron or neuronal cell area results in membrane potentials of a downstream positioned neuron or neuronal cell area. Such membrane potentials are long-term potentiated at least over hours after stimulating the afferent neurons e.g. with a theta burst paradigm. Therefore, LTP is regarded as memory on the cellular level. Electrophysiological LTP measurements on neurons incubated with a test compound in comparison to sham incubated neurons can be used to assess the compounds' potential to enhance memory (See, e.g., Cooke and Bliss, Brain, 2006, 129 (1659), which is hereby incorporated by reference).

There is general agreement that processes underlying memory-formation and learning include structural plasticity of neuronal networks and motility of dendrites or spines (See, e.g., Tada & Sheng, Curr Opin Neurobiol., 2006, 16, 95). Neurite outgrowth is known to be influenced by Rho GTPases, a family of small GTPases with its members Rho, Rac and Cdc42. Rho GTPases are well known for their effects on the actin cytoskeleton and are therefore important regulators of cell motility and synaptic plasticity. Rho in its active GTP-bound form activates Rho kinase (ROCK), which subsequently activates myosin light chain, resulting in the rearrangement of the cytoskeleton and inhibition of axonal growth. It was observed that ROCK inhibitors like Fasudil increase neurite outgrowth in undifferentiated PC12 cells (Zhang et al., Cell Mol Biol Lett., 2006, 11, 12). In order to analyze the effect of a test compound with potential ROCK inhibition ability one can measure the neurite length of primary hippocampal neurons in cell culture in the presence of the test compound in comparison with a control assay without that compound. Alternatively to measuring the increase in length it is possible to determine the increase in complexity (Sholl analysis). A compound that exhibits the ability to stimulate neurite outgrowth can be used for conditions in need of enhancement of cerebral plasticity and cognition.

Familial forms of Alzheimers Disease (AD) and Frontal Temporal Dementia (FTD) and the identification of the causative mutated genes have led to the generation of transgenic animal models for these diseases. The key player in AD is the amyloid precursor protein (APP). Mice overexpressing the mutant APP are the most widely used model to study memory impairment in AD (Ashe, Learn Mem. 2001, 8, 301; Chapman et al., Trends Genet. 2001, 17, 254; Goetz & Ittner, Nat Rev Neurosci. 2008, 9, 532). These mice carry different variants of the amyloid precursor protein (APP) and develop memory deficits over time as it is prominent from AD patients (e.g. animals with the so-called swedish mutation, Tg2576 (Hsiao et al., Science 1996, 274, 99)). These animal models can be utilized to test potential memory-enhancing compounds for their efficacy in an in vivo disease model.

Pathologies or neuropathologies that would benefit from therapeutic and diagnostic applications of this invention include, for example, the following:

diseases of central motor systems including degenerative conditions affecting the basal ganglia (Huntington's disease, Wilson's disease, striatonigral degeneration, corticobasal ganglionic degeneration), Tourette's syndrome, Parkinson's disease, progressive supranuclear palsy, progressive bulbar palsy, familial spastic paraplegia, spinomuscular atrophy, ALS and variants thereof, dentatorubral atrophy, olivo-pontocerebellar atrophy, paraneoplastic cerebellar degeneration, and dopamine toxicity;

diseases affecting sensory neurons such as Friedreich's ataxia, diabetes, peripheral neuropathy, and retinal neuronal degeneration;

diseases of limbic and cortical systems such as cerebral amyloidosis, Pick's atrophy, and Rett syndrome;

neurodegenerative pathologies involving multiple neuronal systems and/or brainstem including Alzheimer's disease, AIDS-related dementia, Leigh's disease, diffuse Lewy body disease, epilepsy, multiple system atrophy, Guillain-Barre syndrome, lysosomal storage disorders such as lipofuscinosis, late-degenerative stages of Down's syndrome, Alper's disease, vertigo as result of CNS degeneration;

pathologies associated with developmental retardation and learning impairments, and Down's syndrome, and oxidative stress induced neuronal death;

pathologies arising with aging and chronic alcohol or drug abuse including, for example, with alcoholism the degeneration of neurons in locus coeruleus, cerebellum, cholinergic basal forebrain; with aging degeneration of cerebellar neurons and cortical neurons leading to cognitive and motor impairments; and with chronic amphetamine abuse degeneration of basal ganglia neurons leading to motor impairments;

pathological changes resulting from focal trauma such as stroke, focal ischemia, vascular insufficiency, hypoxic-ischemic encephalopathy, hyperglycemia, hypoglycemia, closed head trauma, or direct trauma;

pathologies arising as a negative side-effect of therapeutic drugs and treatments (e.g., degeneration of cingulate and entorhinal cortex neurons in response to anticonvulsant doses of antagonists of the NMDA class of glutamate receptor, chemotherapy, antibiotics, etc.); and

learning disabilities such as ADD, ADHD, dyslexia, dysgraphia, dyscalcula, dyspraxia, and information processing disorders.

A number of diseases would benefit from the present invention the pathophysiology of which is related to ROCK 1 and/or 2 kinases. Activities of ROCK in the CNS relate to a number of neuronal functions, such as neurite outgrowth and retraction, but also to neuronal apoptosis. In the adult CNS axonal re-growth after injuries is inhibited by myelin-associated signals (such as Nogo, MAG). ROCK is involved in this phenomenon. Consequently, inhibition of ROCK activity helps in overcoming these inhibitory signals, and is therefore beneficial for axonal rewiring in spinal cord injury, brain injuries, or post-stroke recovery. In addition, ROCK is involved in apoptotic pathways. Inhibition of ROCK therefore should be beneficial to diseases associated with (apoptotic) cell death in the CNS or PNS (peripheral nervous system). Typical diseases are stroke, brain injury, cerebral hemorrhage, and neurodegenerative diseases (such as Amyotrophic lateral sclerosis, Huntington's disease, Parkinson's disease, hereditary ataxias, hereditary metabolic disorders of the CNS).

In the cardiovascular system ROCK has a prominent activity on regulating vascular tone. In addition, involvement in smooth muscle or cardiac muscle apoptotic pathways has been noted. Consequently, ROCK inhibition should be useful for diseases with deregulated vascular tone or resistance or compliance. Such diseases include, for example: vasospasms following subarachnoid hemorrhage, angina pectoris (preferentially Prinzmetal's or vasospastic angina), heart failure-associated diseases (e.g. due to vascular resistance and constriction), myocardial infarction, pulmonary arterial hypertension essential hypertension, atherosclerosis and aortic stiffness, and peripheral vascular diseases like Reynaud's phenomenon, and erectile dysfunctions. Metastasis of cancer cells is dependent on cell migration, a complex process regulated spatially as well as temporally by the Rho family members GTPases Rho, Rac and Cdc42. In particular, the Rho effectors ROCK I and II are involved in these processes. For example, membrane blebbing has been shown to be induced by ROCK and amoeboid-like movement is completely dependent on the interaction between Rho and ROCK. Therefore, inhibition of ROCK may be beneficial for the treatment of (metastasizing) cancer (e.g. prostate, breast, lung, colon cancer, glioblastoma, sarcomatous tumours, melanoma among others).

### I. DEFINITIONS

Memory systems can be classified broadly into four main types: episodic, semantic, working, and procedural (Hwang, D. Y. & Golby, A. J. Epilepsy Behav (2005); Yancey, S. W. & Phelps, E. A. J Clin Exp Neuropsychol 23, 32-48 (2001)). Episodic memory refers to a system that records and retrieves autobiographical information about experiences that occurred at a specific place and time. The semantic memory system stores general factual knowledge unrelated to place and time (e.g. the capital of Arizona). Working memory involves the temporary maintenance and usage of information while procedural memory is the action of learning skills that operate automatically and, typically, unconsciously. Episodic, semantic, and working memory are explicit (absolute) and declarative (explanatory) in nature while procedural memory can be either explicit or implicit, but is always nondeclarative (Tulving, E. Oxford University Press, New York, 1983); Budson, A. E., Price, B. H. Encyclopedia of Life Sciences (Macmillan, Nature Publishing Group, London, 2001); Budson, A. E. & Price, B. H. N Engl J Med 352, 692-9 (2005); Hwang, D. Y. & Golby, A. J Epilepsy Behav 8, 115-26 (2006)).

Normal aging states and disease states that impair memory include but are not limited to neurodegenerative disorders, head and brain trauma, genetic disorders, infectious disease, inflammatory disease, medication, drug and alcohol disorders, cancer, metabolic disorders, mental retardation, and learning and memory disorders, such as age related memory loss and age-associated memory impairment (AAMI), Alzheimer's disease, tauopathies, PTSD (post traumatic stress syndrome), mild cognitive impairment, ALS, Huntington's chorea, amnesia, B1 deficiency, schizophrenia, depression and bipolar disorder, stroke, hydrocephalus, subarachnoid hemorrhage, vascular insufficiency, brain tumor, epilepsy, Parkinson's disease, cerebral microangiopathy (Meyer, R. C., et al. Aim N Y Acad Sci 854, 307-17 (1998); Barrett, A. M. Postgrad Med 117, 47-53 (2005); Petersen, R. C. J Intern Med 256, 183-94 (2004); Calkins, M. E., et al. Am J Psychiatry 162, 1963-6 (2005)), pain medication, chemotherapy (“chemobrain”), oxygen deprivation, e.g, caused by a heart-lung machine, anesthesia, or near drowning, dementia (vascular, frontotemporal, Lewy-body, semantic, primary progressive aphasia, Pick's), progressive supranuclear palsy, corticobasal degeneration, Hashimoto encephalopathy, ADD, ADHD, dyslexia and other learning disabilities, Down syndrome, fragile X syndrome, Turner's syndrome, and fetal alcohol syndrome, for example. Memory deficits also may occur as sequelae of surgical procedures, especially cardiac surgery, and surgery of the large vessels. In addition to disease, progressive memory loss is a normal byproduct of the aging process.

The term mild cognitive impairment (MCI) is used to refer to a transitional zone between normal cognitive function and the development of clinically probable AD (Winblad, B. et al. J Intern Med 256, 240-6 (2004)). A variety of criteria have been utilized to define MCI, however they essentially have two major themes: (1) MCI refers to non-demented patients with some form of measurable cognitive defects and (2) these patients represent a clinical syndrome with a high risk of progressing to clinical dementia.

The phrase “improving learning and/or memory” refers to an improvement or enhancement of at least one parameter that indicates learning and memory. Improvement or enhancement is change of a parameter by at least 10%, optionally at least about 20%, at least about 30%, at least about 40%, at least about 50%, at least about 60%, at least about 70%, at least about 80%, at least about 90%, at least about 100%, at least about 150%, at least about 200%, etc. The improvement of learning and memory can be measured by any methods known in the art. For example, compounds described herein that improve learning and memory can be screened using Morris water maze (see, e.g., materials and methods section). See also, Gozes et al., Proc. Natl. Acad. Sci. USA 93:427-432 (1996), radial arm maze, object recognition, open field, Sacktor-disc etc. Memory and learning can also be screened using any of the methods described herein or other methods that are well known to those of skill in the art, e.g., the Randt Memory Test, the Wechsler Memory Scale, the Forward Digit Span test, or the California Verbal Learning Test.

The term “spatial learning” refers to learning about one's environment and requires knowledge of what objects are where. It also relates to learning about and using information about relationships between multiple cues in environment. Spatial learning in animals can be tested by allowing animals to learn locations of rewards and to use spatial cues for remembering the locations. For example, spatial learning can be tested using a radial arm maze (i.e., learning which arm has food) or a Morris water maze (i.e., learning where the platform is). To perform these tasks, animals use cues from test room (positions of objects, odors, etc.). In human, spatial learning can also be tested. For example, a subject can be asked to draw a picture, and then the picture is taken away. The subject is then asked to draw the same picture from memory. The latter picture drawn by the subject reflects a degree of spatial learning in the subject.

Learning disabilities is a general term that refers to a heterogeneous group of disorders manifested by significant difficulties in the acquisition and use of listening, speaking, reading, writing, reasoning, or mathematical abilities. Learning disabilities include ADD, ADHD, dyslexia, dysgraphia, dyscalcula, dyspraxia, and information processing disorders.

As used herein, “administering” refers to oral administration, administration as a suppository, topical contact, parenteral, intravenous, intraperitoneal, intramuscular, intralesional, oral, intranasal or subcutaneous administration, intrathecal administration, or the implantation of a slow-release device e.g., a mini-osmotic pump, to the subject.

As used herein, the term “alkyl” refers to a straight or branched, saturated, aliphatic group having the number of carbon atoms indicated. For example, C₁-C₆ alkyl includes, but is not limited to, methyl, ethyl, propyl, butyl, pentyl, hexyl, iso-propyl, iso-butyl, sec-butyl, tert-butyl, etc.

As used herein, the term “halogen” refers to fluorine, chlorine, bromine and iodine.

As used herein, the term “heterocycle” refers to a ring system having from 5 to 8 ring members and 2 nitrogen heteroatoms. For example, heterocycles useful in the present invention include, but are not limited to, pyrazolidine, imidazolidine, piperazine and homopiperazine. The heterocycles of the present invention are N-linked, meaning linked via one of the ring heteroatoms.

As used herein, the term “hydrate” refers to a compound that is complexed to at least one water molecule. The compounds of the present invention can be complexed with from 1 to 10 water molecules.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly, acid addition salts, such as of mineral acids, organic carboxylic and organic sulfonic acids, e.g., hydrochloric acid, methanesulfonic acid, maleic acid, also are possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

As used herein, the term “subject” refers to animals such as mammals, including, but not limited to, primates (e.g., humans), cows, sheep, goats, horses, dogs, cats, rabbits, rats, mice and the like. Preferably, the subject is a human.

As used herein, the terms “therapeutically effective amount” or “therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

### II. METHODS OF USE

Methods are provided for improving memory and learning by the administration of a compound of Formula I, salts, hydrates and solvates thereof. As indicated in the examples below, the inventive compounds are used to enhance memory, improve neural plasticity, and/or treat Alzheimer's disease. The compounds can be administered orally, parenterally, or nasally, for example. For long term administration, lower doses can be used. The compounds according to the invention can be used in combination with other drugs to treat disease states or improve learning and memory. Furthermore, compounds of the inventions can be used as specific and potent ROCK inhibitors. Therefore, they are suitable for the treatment of ROCK related diseases, e.g. vasospasms following subarachnoid hemorrhage.

In one aspect, compounds of the invention are particularly potent and highly specific ROCK inhibitors. The compounds also show inhibitory effect particularly for the PIM kinases and for the IRAK1 kinase.

The PIM kinases are of high medical relevance. PIM kinases (Pim-1, -2, and -3) are highly conserved serine-threonine kinases belonging to the CAMK (calmodulin-dependent protein kinase-related) group that are key regulators in many signalling pathways implicated in cancer. Pim-1 was first identified with c-myc as a frequent proviral insertion site in Moloney murine leukemia virus-induced T-cell lymphomas. When expressed, PIM kinases are strong survival factors and can induce progression of the cell cycle, inhibition of apoptosis, and modulation of other signal transduction pathways. Knockout mice for all three pim genes develop normally but display reduced body size owing to decreased cell number in virtually all tissues. Pim kinases contribute to both cell proliferation and survival and thus provide a selective advantage in tumorigenesis. A number of proteins are phosphorylated by Pim kinases, such as transcriptional repressors (HP1), activators such as NFATc1 and c-Myb, co-activators (p100), as well as regulators of the cell cycle, such as p21WAF1/CIP1, Cdc25A phosphatase, and the kinase C-TAK1/MARK3/Par1A. PIM inhibitors can therefore induce cell death in cancer cells expressing PIM kinases and promote sensitivity of cancer cells to treatment with other targeted and chemotherapy drugs. Compounds of the invention which exhibit a particular specific inhibitory effect on PIM kinases beside their inhibitory effect on ROCK therefore have broad therapeutic potential as a single agent as well as in combination with other agents (e.g. chemotherapeutic drugs and regimes known to anyone skilled in the art, such as imatinib mesylate, mechlorethamine, cyclophosphamide, chlorambucil, cisplatin, carboplatin, oxaliplatin, azathioprine, mercaptopurine, doxorubicine, epirubicin, bleomycin, dactinomycin, vincristine, vinblastine, vinorelbine, vindesine, etoposide, teniposide, podophyllotoxin, paclitaxel, irinotecan, topotecan, melphalan, busulfan, capecitabine and combination thereof).

As PIM kinases contribute to many malignancies including prostate adenocarcinomas, pancreatic carcinoma, breast cancer, lung cancer, melanoma, liver carcinoma, gastric adenocarcinoma, diffuse large cell lymphomas, as well as several types of leukemias and other hematological malignancies, PIM kinase inhibition is useful for the treatment of a large number of malignancies. In particular, PIM kinase inhibition is useful for the treatment of leucemias ALL, CLL, AML, or CML, and Hodgkin- and Non-Hodgkin Lymphomas, mantle-cell lymphoma, Burkitt's lymphoma, and myeloproliferative disease (Amaravadi et al., J Clin Invest 2005, 115, 2618; Chiang et al., Int J Oral Maxillofac Surg. 2006, 35, 740; Dai et al. Acta Pharmacol Sin. 2005, 26, 364; Hu et al., J Clin Invest. 2009, 119, 362; Popivanova et al., Cancer Sci. 2007, 98, 321; Reiser-Erkan et al., Cancer Biol Ther. 2008, 7, 1352; Shah et al., Eu J Cancer 2008, 44, 2144; Tong et al., Bioorg Med Chem. Lett. 2008, 18, 5206; Wang et al., J Vet Sci. 2001, 2, 167; Xia et al., J Med. Chem. 2009, 52, 74; Zemskova et al., J Biol. Chem. 2008, 283, 20635). Indeed a number of clinical trials are being initiated with compounds that target PIM kinases, such as SGI-1776. Therefore, compounds according to the invention which exhibit a particular specific inhibitory effect on PIM kinases are a new, highly attractive and desirable drug candidate. A specific advantage is the ran-PIM activity, and the high specificity of the named compounds.

The interleukin-1 receptor-associated kinase 1 (IRAK1) is a putative serine/threonine kinase that associates with the interleukin-1 receptor (IL1R) upon stimulation. This gene is partially responsible for IL1-induced upregulation of the transcription factor NF-kappa B. IRAK genes are linked with diverse diseases such as infection, atherosclerosis, sepsis, auto-immune diseases, and cancer. IRAKs are involved in multiple signaling networks and diverse tissues and cells such as adipocytes, hepatocytes, muscle cells, endothelial cells, and epithelial cells. Conceivably, these molecules pose particular targets for designing new therapeutic strategies for various human inflammatory diseases (like MS, inflammatory bowel disease, Reiter's disease and Rheumatoid arthritis). Evidence suggests that interleukin-1 receptor-associated kinase-1 (IRAK1) plays a fundamental role in the toll-like receptor pathway (TLR) and in the regulation of the transcription factor NF-kappa B. Variations in human IRAK genes have been found to be linked with various human inflammatory diseases. Deletion of the IRAK-1 gene in mice decreases the risk of Experimental Autoimmune Encephalomyelitis (EAE) (Deng et al., J. Immunol. 2003, 170, 2833). Moreover, the IRAK-1 protein has been shown to be constitutively activated/sumoylated and localizes in cell nucleus in leukocytes from human atherosclerosis patients (Huang et al., J Biol. Chem. 2004, 279, 51697). Furthermore, human population-based study indicates that genetic variation in IRAK-1 gene correlates with the severity of atherosclerosis and serum C reactive protein levels (Lakoski et al., Exp Mol. Pathol. 2007, 82, 280).

There are two IRAK-1 haplotypes, and a rare variant haplotype (˜10% of human population) contains three exon single nucleotide polymorphisms (SNPs). Humans harboring the variant IRAK-1 gene tend to have higher serum CRP levels and are at higher risk for diabetes and hypertension. IRAK-1 gene variation also is linked to a risk of sepsis. Arcaroli et al. demonstrated that sepsis patients with the rare variant IRAK-1 haplotype have increased incidence of shock, prolonged requirement for mechanical ventilatory support, and greater 60-day mortality (Arcaroli et al., Am J Respir Crit. Care Med. 2006, 173, 1335).

The interleukin receptor associated kinase-M (IRAK-M) is a NF-kappaB-mediated, negative regulator of Toll-like receptor (TLR) signaling. A functional mutation in a negative regulator might induce impaired endotoxin tolerance and increased inflammatory responses. Impaired negative regulation of the TLR-signaling pathway might be partly responsible for the development of inflammatory bowel diseases (IBD). Important other diseases where IRAK inhibition may be beneficial include stroke, spinal cord injury, brain trauma, Guillain-Barré syndrome. Especially interesting are autoimmune diseases, but also inflammatory conditions linked to infection.

In another aspect, methods are provided for treating a patient for anxiety, depression, bipolar disorder, unipolar disorder, and post-traumatic stress disorder by administering to said patient a therapeutically effective amount of a compound according to the formula:

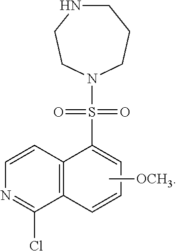

In one example, the compound is 1-(1-chloro-8-methoxy-5 isoquinoline-sulfonyl)homopiperazine.

In other aspects, methods are provided for treating conditions related to a kinase selected of the group consisting of CSNK1E, CSNK1A1L, CSNK1D, MERTK, SLK, IRAK1, STK10, MAPK12, PHKG2, MAPK11, MET, AXL, STK32B, AURKC, CLK3, RPS6KA6, PDGFRB, KDR, CDK2 in a subject, the method comprising administering to a patient in need thereof, a therapeutically effective amount of a compound of the formula:

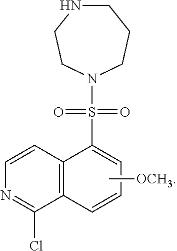

### III. COMPOUNDS

The present invention provides compounds of Formula I:

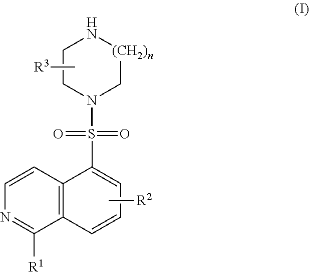

wherein R¹ is a member selected from the group consisting of hydrogen, C₁₋₆ alkyl, hydroxy, and halogen. In one embodiment, R¹ is selected from the group consisting of hydrogen and C₁₋₆ alkyl.

R² is a member selected from the group consisting of C₁₋₆ alkyl, halogen, —C(O)—R⁴, C₁₋₆ alkoxy, C₁₋₆ haloalkyl, —C(O)N(R⁴)R⁴, —N(R⁴)—C(O)—R⁴, —N(R⁴)R⁴, and —C(O)OR⁴, whereas R² is localized at position 6, 7, or 8, such as at position 8 of the isoquionline moiety.

R³ is a member selected from the group consisting of hydrogen, and C₁₋₆ alkyl.

Each R⁴ is independently a member selected from the group consisting of hydrogen, C₁₋₆ alkyl and C₃₋₈ cycloalkyl.

Subscript n is 0, 1, or 2, preferably 1 or 2.

In some embodiments where R¹, R², R³, or R⁴ is an alkyl, alkoxy, or haloalkyl group, the group is selected from C₁₋₃ alkyl, C₁₋₃ alkoxy, and C₁₋₃ haloalkyl, respectively.

The compounds of formula I can also be salts, hydrates and solvates thereof.

In general, compounds of Formula I, and their salts and hydrates, can be prepared using well-established methodologies and are based on the common knowledge of one skilled in the art. These are described, for instance, in U.S. Pat. Nos. 4,678,783 and 5,942,505 and European Patent No. 187,371, which are incorporated in their entireties herein by reference. More specific methodologies for representative compounds of the invention are presented in detail below.

In some embodiments, compounds according to the invention are of the Formula I, wherein R¹ is a member selected from the group consisting of hydrogen, C₁₋₆ alkyl, hydroxy, and halogen, such as hydrogen, halogen and C₁₋₆ alkyl, and in some embodiments hydrogen and C₁₋₆ alkyl; R² is C₁₋₆ alkyl whereas R² is localized at position 6, 7, or 8, such as at position 8 of the isoquionline moiety; R³ is a member selected from the group consisting of hydrogen, and C₁₋₆ alkyl; and n is 0, 1, or 2, such as 1 or 2. In these embodiments when R¹, R², R³, or R⁴ is an alkyl, alkoxy, or haloalkyl group, said group is a C₁₋₃ alkyl, C₁₋₃ alkoxy, or C₁₋₃ haloalkyl, respectively.

In one embodiment, the compound is of the Formula:

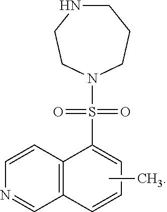

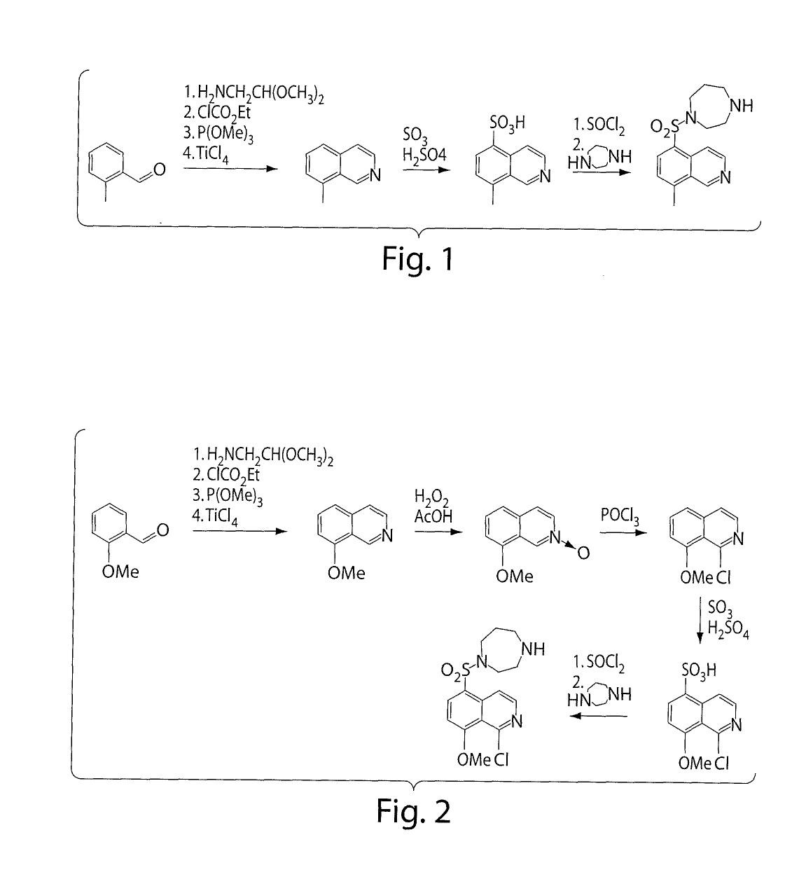

An exemplary compound is 1-(8-methyl-5 isoquinoline-sulfonyl)homopiperazine. A illustrative method for synthesizing the compound is depicted in FIG. 1. Related compounds can be prepared analogously.

In other embodiments, compounds according to the invention are of the Formula I, wherein R¹ is a member selected from the group consisting of hydrogen, C₁₋₆ alkyl, hydroxy, and halogen, such as from the group consisting of hydrogen, halogen and C₁₋₆ alkyl, for instance hydrogen and C₁₋₆ alkyl; R² is C₁₋₆ alkoxy whereas R² is localized at position 6, 7, or 8, such as at position 8 of the isoquionline moiety; R³ is a member selected from the group consisting of hydrogen, and C₁₋₆ alkyl; and n is 0, 1, or 2, such as 1 or 2. In some embodiments where R¹, R², R³, or R⁴ is an alkyl, alkoxy, or haloalkyl group, the group is a C₁₋₃ alkyl, C₁₋₃ alkoxy, or C₁₋₃ haloalkyl, respectively. Such compounds are particularly potent and highly specific ROCK inhibitors. Therefore, the method of use of the compounds as ROCK inhibitors is also an embodiment of the invention. Beside the ROCK inhibition compounds of this group show inhibitory effect particularly only for the PIM kinases and for the IRAK1 kinase. Therefore, the method of use of the compounds as PIM kinase and/or IRAK1 kinase is a further embodiment of the invention.

In one embodiment, the compound is of the Formula:

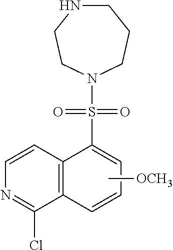

### or of the Formula:

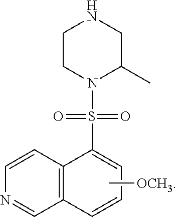

The inventive compounds are particularly potent ROCK inhibitors. In addition, inventive compounds specifically can inhibit PIM and IRAK1 kinases. In some embodiments, therefore, the inventive compounds can be used to inhibit ROCK or PIM kinases or IRAK1 kinases.

Another exemplary compound is 1-(1-chloro-8-methoxy-5 isoquinoline-sulfonyl)homopiperazine. A method of synthesizing the compound depicted in FIG. 2. Related compounds can be prepared analogously. 1-(1-chloro-8-methoxy-5 isoquinoline-sulfonyl)homopiperazine is a particularly potent and highly specific ROCK inhibitor. Therefore, the method of use of this compound as ROCK inhibitors is also an embodiment of the invention. Besides ROCK inhibition, this compound shows selective inhibitory effect particularly for PIM kinases and for IRAK1 kinase. Therefore, methods of using of this compound as a inhibitor of PIM kinase and/or IRAK1 kinase is a further embodiment of the invention.

In some embodiments, compounds according to the invention are of the Formula I, wherein R¹ is a member selected from the group consisting of hydrogen, C₁₋₆ alkyl, hydroxy, and halogen, such as from the group consisting of hydrogen, halogen and C₁₋₆ alkyl, such as hydrogen and C₁₋₆ alkyl; R² is —C(O)—R⁴; wherein R⁴ is a member selected from the group consisting of hydrogen, C₁₋₆ alkyl and C₃₋₈ cycloalkyl; and whereas R² is localized at position 6, 7, or 8, such as at position 8 of the isoquionline moiety; R³ is a member selected from the group consisting of hydrogen, and C₁₋₆ alkyl; and n is 0, 1, or 2, such as 1 or 2. In some embodiments where R¹, R³, or R⁴ is an alkyl, alkoxy, or haloalkyl group, the group is a C₁₋₃ alkyl, C₁₋₃ alkoxy, or C₁₋₃ haloalkyl, respectively.

In one embodiment, the compound is of the Formula:

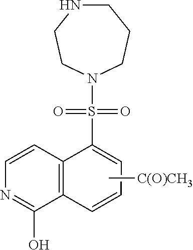

### or of the Formula:

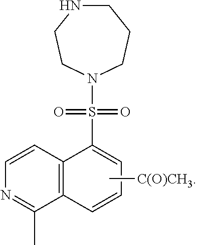

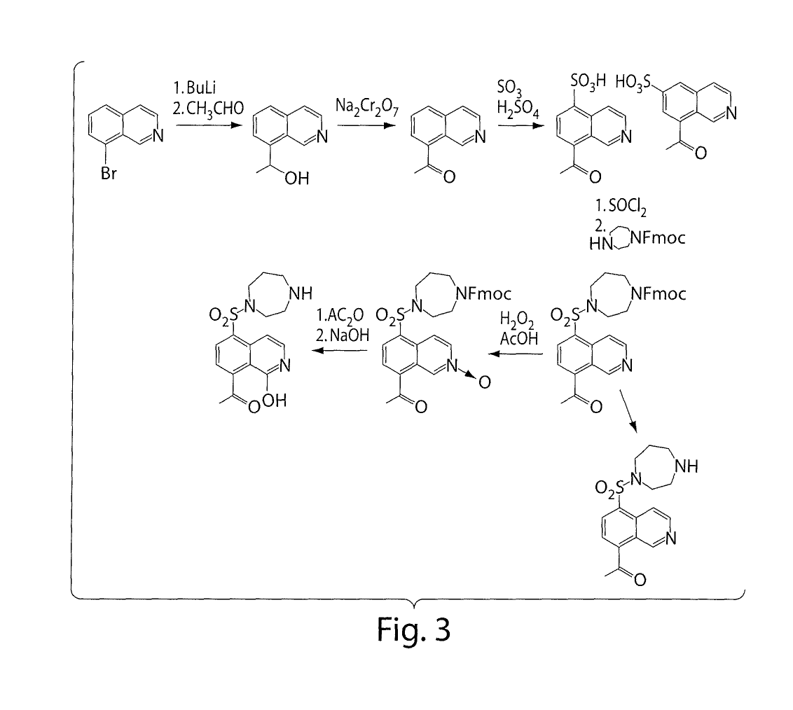

Another exemplary compound is 1-(1-hydroxy-8-acetyl-5 isoquinoline-sulfonyl)homopiperazine or 1-(8-acetyl-5 isoquinoline-sulfonyl)homopiperazine. An illustrative synthesis of the compound is depicted in FIG. 3. Related compounds can be prepared analogously.

In other embodiments, compounds according to the invention are of the Formula I, wherein R¹ is a member selected from the group consisting of hydrogen, C₁₋₆ alkyl, hydroxy, and halogen, such as from the group consisting of hydrogen, halogen and C₁₋₆ alkyl, such as hydrogen and C₁₋₆ alkyl; R² is —C(O)—N(R⁴)R⁴; wherein R⁴ is a member selected from the group consisting of hydrogen, C₁₋₆ alkyl and C₃₋₈ cycloalkyl; and whereas R² is localized at position 6, 7, or 8, such as at position 8 of the isoquionline moiety; R³ is a member selected from the group consisting of hydrogen, and C₁₋₆ alkyl; and n is 0, 1, or 2, such as 1 or 2. In some embodiments where R¹, R³, or R⁴ is an alkyl, alkoxy, or haloalkyl group, the group is a C₁₋₃ alkyl, C₁₋₃ alkoxy, or C₁₋₃ haloalkyl, respectively.

In some embodiments, the compound is of the Formula:

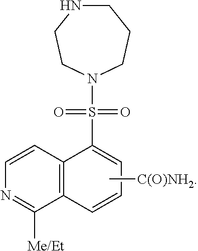

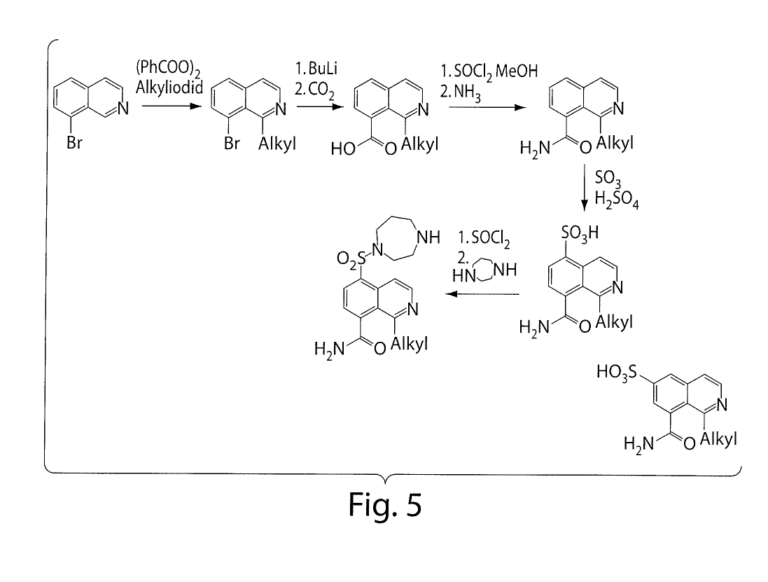

Another illustrative compound is 1-(1-methyl-8-carboxamide-5 isoquinoline-sulfonyl)homopiperazine or 1-(1-ethyl-8-carboxamide-5 isoquinoline-sulfonyl)homopiperazine. An illustrative synthesis is depicted in FIG. 5. Related compounds can be prepared analogously.

In other embodiments, compounds according to the invention are of the Formula I, wherein R¹ is a member selected from the group consisting of hydrogen, C₁₋₆ alkyl, hydroxy, and halogen, such as from the group consisting of hydrogen, halogen and C₁₋₆ alkyl, such as hydrogen and C₁₋₆ alkyl; R² is —N(R⁴)—C(O)—R⁴; wherein R⁴ is a member selected from the group consisting of hydrogen, C₁₋₆ alkyl and C₃₋₈ cycloalkyl; and whereas R² is localized at position 6, 7, or 8, such as at position 8 of the isoquionline moiety; R³ is a member selected from the group consisting of hydrogen, and C₁₋₆ alkyl; and n is 0, 1, or 2, such as 1 or 2. Where, for instance, R¹, R³, or R⁴ is an alkyl, alkoxy, or haloalkyl group, the group is a C₁₋₃ alkyl, C₁₋₃ alkoxy, or C₁₋₃ haloalkyl, respectively.

In some embodiments, the compound is of the Formula:

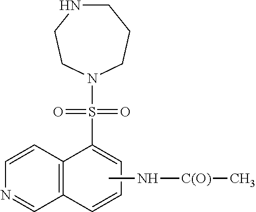

### or of the Formula:

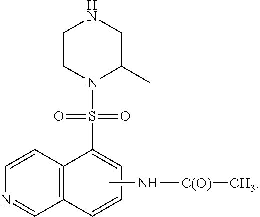

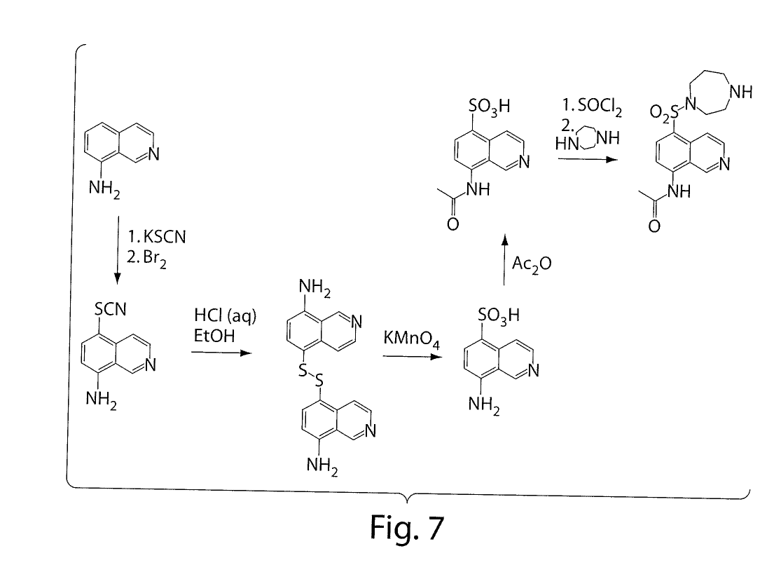

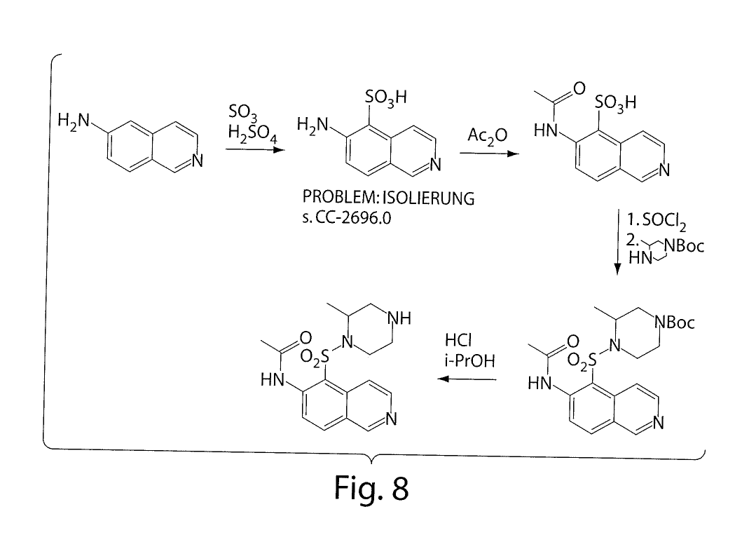

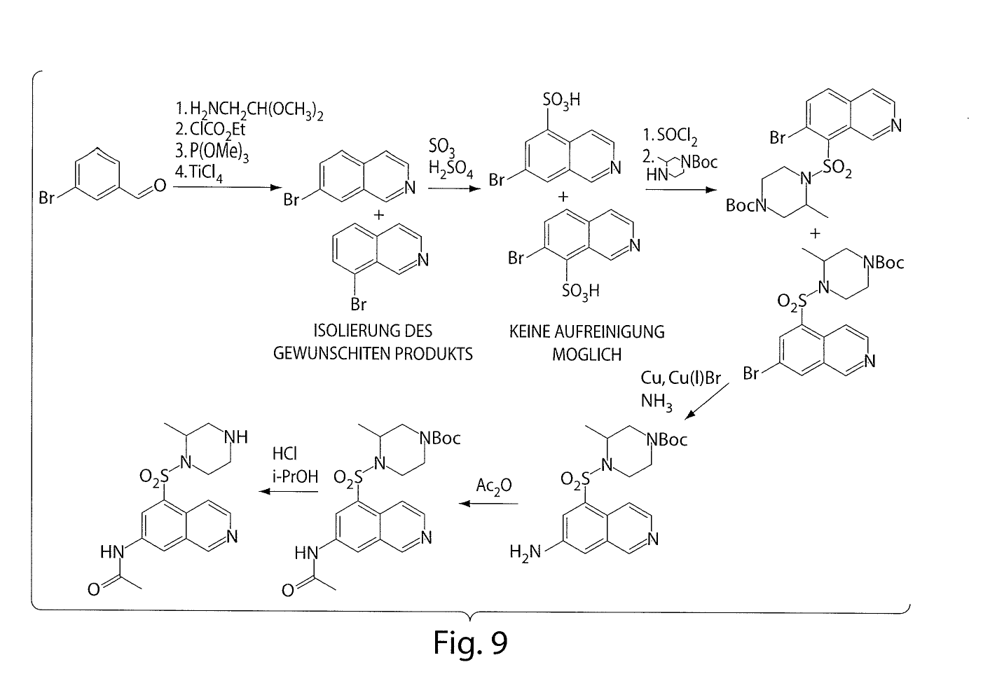

Exemplary synthetic pathways are depicted in FIG. 7, FIG. 8, and FIG. 9. Related compounds can be prepared analogously. Another illustrative compound is 1-(8-aminoacetyl-5 isoquinoline-sulfonyl)homopiperazine.

In some embodiments, compounds according to the invention are of the Formula I, wherein R¹ is a member selected from the group consisting of hydrogen, C₁₋₆ alkyl, hydroxy, and halogen, such as from the group consisting of hydrogen, halogen and C₁₋₆ alkyl, such as hydrogen and C₁₋₆ alkyl; R² is —N(R⁴)—R⁴; wherein R⁴ is a member selected from the group consisting of hydrogen, C₁₋₆ alkyl and C₃₋₈ cycloalkyl; and whereas R² is localized at position 6, 7, or 8, such as at position 8 of the isoquionline moiety; R³ is a member selected from the group consisting of hydrogen, and C₁₋₆ alkyl; and n is 0, 1, or 2, such as 1 or 2. Where, for example, R¹, R³, or R⁴ is an alkyl, alkoxy, or haloalkyl group, the group is a C₁₋₃ alkyl, C₁₋₃ alkoxy, or C₁₋₃ haloalkyl, respectively.

In some embodiments, the compound is of the Formula:

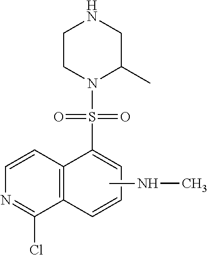

### or of Formula:

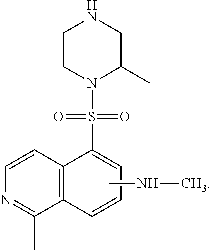

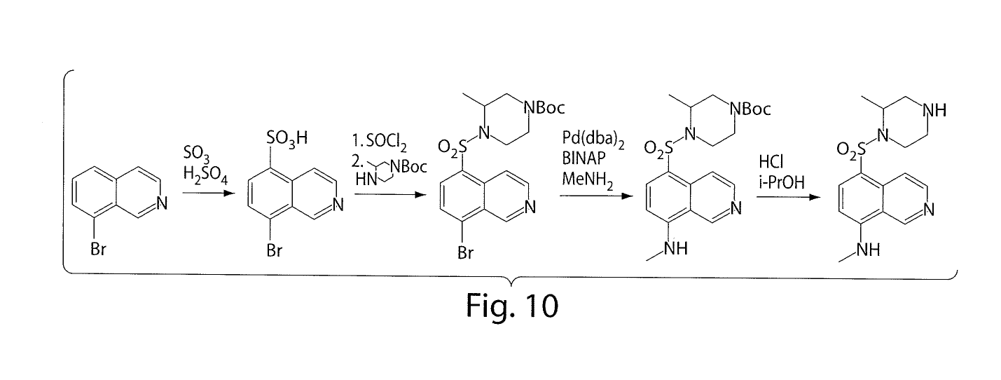

An illustrative compound is 1-(8-aminomethyl-5 isoquinoline-sulfonyl) 2-methyl-piperazine. One synthetic pathway is depicted in FIG. 10. Related compounds can be prepared analogously.

In other embodiments, compounds according to the invention are of the Formula I, wherein R¹ is a member selected from the group consisting of hydrogen, C₁₋₆ alkyl, hydroxy, and halogen, such as from the group consisting of hydrogen, halogen and C₁₋₆ alkyl, such as hydrogen and C₁₋₆ alkyl; R² is a halogen, preferably a chlorine; whereas R² is localized at position 6, 7, or 8, such as at position 8 of the isoquionline moiety; R³ is a member selected from the group consisting of hydrogen, and C₁₋₆ alkyl; and n is 0, 1, or 2, such as 1 or 2. Where, for example, R¹, R³, or R⁴ is an alkyl, alkoxy, or haloalkyl group, the group is a C₁₋₃ alkyl, C₁₋₃ alkoxy, or C₁₋₃ haloalkyl, respectively.

In some embodiments, the compound is of the Formula:

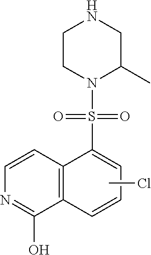

### or of the Formula:

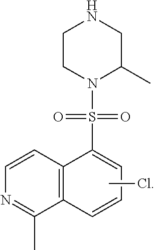

In other embodiments, compounds according to the invention are of the Formula I, wherein R¹ is a member selected from the group consisting of hydrogen, C₁₋₆ alkyl, hydroxy, and halogen, such as from the group consisting of hydrogen, halogen and C₁₋₆ alkyl, such as hydrogen and C₁₋₆ alkyl; R² is C₁₋₆ haloalkyl; whereas R² is localized at position 6, 7, or 8, such as at position 8 of the isoquionline moiety; R³ is a member selected from the group consisting of hydrogen, and C₁₋₆ alkyl; and n is 0, 1, or 2, such as 1 or 2. Where, for instance, R¹, R², R³, or R⁴ is an alkyl, alkoxy, or haloalkyl group, the group is a C₁₋₃ alkyl, C₁₋₃ alkoxy, or C₁₋₃ haloalkyl, respectively.

In some embodiments, the compound is of the Formula:

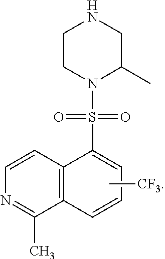

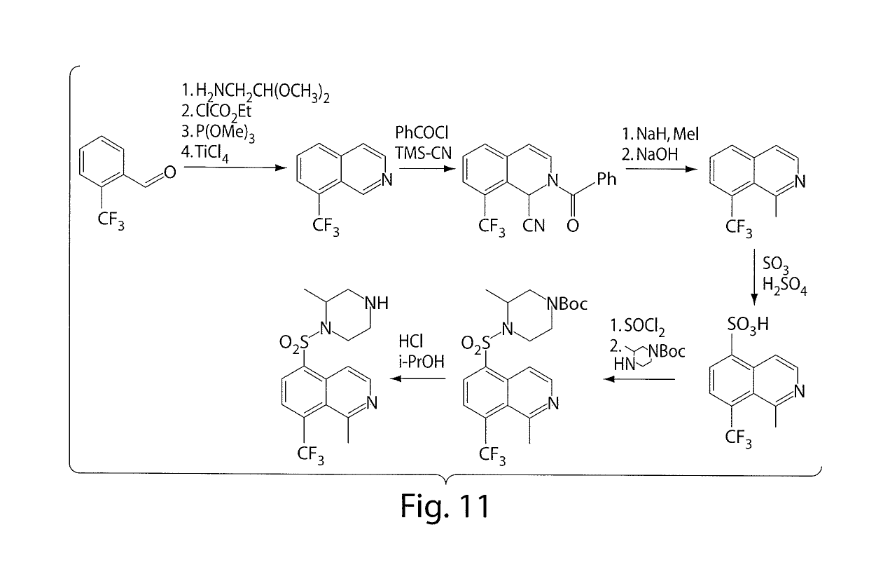

Another illustrative compound is 1-(1-methyl-8-trifluoromethyl-5 isoquinoline-sulfonyl) 2-methyl-piperazine. An exemplary synthetic scheme is depicted in FIG. 11. Related compounds can be prepared analogously.

In some embodiments, compounds according to the invention are of the Formula I, wherein R¹ is a member selected from the group consisting of hydrogen, C₁₋₆ alkyl, hydroxy, and halogen, such as from the group consisting of hydrogen, halogen and C₁₋₆ alkyl, such as hydrogen and C₁₋₆ alkyl; R² is —C(O)OR⁴; wherein R⁴ is a member selected from the group consisting of hydrogen, C₁₋₆ alkyl and C₃₋₈ cycloalkyl; and whereas R² is localized at position 6, 7, or 8, such as at position 8 of the isoquionline moiety; R³ is a member selected from the group consisting of hydrogen, and C₁₋₆ alkyl; and n is 0, 1, or 2, preferably 1 or 2. Where, for instance, R¹, R², R³, or R⁴ is an alkyl, alkoxy, or haloalkyl group, the group is a C₁₋₃ alkyl, C₁₋₃ alkoxy, or C₁₋₃ haloalkyl, respectively.

### IV. FORMULATIONS FOR IMPROVING MEMORY AND LEARNING

The compounds of the present invention can be formulated in a variety of different manners known to one of skill in the art. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 20^{th} ed., 2003, supra). Effective formulations include oral and nasal formulations, formulations for parenteral administration, and compositions formulated for with extended release.

Formulations suitable for oral administration can consist of (a) liquid solutions, such as an effective amount of a compound of the present invention suspended in diluents, such as water, saline or PEG 400; (b) capsules, sachets, depots or tablets, each containing a predetermined amount of the active ingredient, as liquids, solids, granules or gelatin; (c) suspensions in an appropriate liquid; (d) suitable emulsions; and (e) patches. The pharmaceutical forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents. Preferred pharmaceutical preparations can deliver the compounds of the invention in a sustained release formulation.

Pharmaceutical preparations useful in the present invention also include extended-release formulations. In some embodiments, extended-release formulations useful in the present invention are described in U.S. Pat. No. 6,699,508, which can be prepared according to U.S. Pat. No. 7,125,567, both patents incorporated herein by reference.

The pharmaceutical preparations are typically delivered to a mammal, including humans and non-human mammals. Non-human mammals treated using the present methods include domesticated animals (i.e., canine, feline, murine, rodentia, and lagomorpha) and agricultural animals (bovine, equine, ovine, porcine).

In practicing the methods of the present invention, the pharmaceutical compositions can be used alone, or in combination with other therapeutic or diagnostic agents.

### V. ADMINISTRATION FOR IMPROVING MEMORY AND LEARNING

The compounds of the present invention can be administered as frequently as necessary, including hourly, daily, weekly or monthly. The compounds utilized in the pharmaceutical method of the invention are administered at the initial dosage of about 0.0001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of disease diagnosed in a particular patient. The dose administered to a patient, in the context of the present invention should be sufficient to effect a beneficial therapeutic response in the patient over time. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot, implanted micro pump or via a patch.

The pharmaceutical compositions can be administered to the patient in a variety of ways, including topically, parenterally, intravenously, intradermally, subcutaneously, intramuscularly, colonically, rectally or intraperitoneally. Preferably, the pharmaceutical compositions are administered parenterally, topically, intravenously, intramuscularly, subcutaneously, orally, or nasally, such as via inhalation.

In practicing the methods of the present invention, the pharmaceutical compositions can be used alone, or in combination with other therapeutic or diagnostic agents. The additional drugs used in the combination protocols of the present invention can be administered separately or one or more of the drugs used in the combination protocols can be administered together, such as in an admixture. Where one or more drugs are administered separately, the timing and schedule of administration of each drug can vary. The other therapeutic or diagnostic agents can be administered at the same time as the compounds of the present invention, separately or at different times.

### VI. EXAMPLES

### Example 1

### Preparation of 1-(8-methyl-5 isoquinoline-sulfonyl)homopiperazine

1-(8-methyl-5 isoquinoline-sulfonyl)homopiperazine was manufactured according to FIG. 1. 20 g 2-Methylbenzaldehyde and 17.5 g aminoacetaldehyde dimethyl acetal were dissolved in 200 ml toluene and 3 h boiled using a reflux condenser. Solvent was removed and residue dissolved in 120 ml dry THF. 15.9 ml ethyl chloroformate was added dropwise at 0° C. with subsequent 5 min stirring at 0° C. After warming to ambient temperature 19.6 ml trimethyl phosphite were added dropwise with subsequent stirring over night. Solvent was distilled off and residue was concentrated two times with toluene to remove residual trimethyl phosphate. The oily residue was dissolved under argon atmosphere in 200 ml dry dichloromethane. Subsequently, 110 ml titanium tetrachloride were added carefully. The solution was boiled over night using a reflux condenser and subsequently carefully poured in ice. pH value was adjusted to 8 using 10% sodium hydroxide solution. Aqueous phase was extracted three times with 500 ml dichloromethane and combined organic phases were washed with water and saturated sodium chloride solution and subsequently dried over sodium sulphate and concentrated which yielded to 11.8 g of 8-methyl-isoquinoline which is an yellow oil. The yield was 11.8 g of 8-methyl-5 isoquinoline which is an yellow oil.

7.3 g of 8-methyl-5 isoquinoline were dissolved in 50 ml ice cold sulphuric acid with subsequent adding of 50 ml oleum with further cooling. After stirring 3 h at 80° C. the solution was poured on ice water and the precipitate was filtered, suspended in ethyl ether, filtered again, washed with ether and vacuum dried which resulted in 7.4 g of 8-methyl-5 isoquinoline-sulfonic acid, which is a brownish solid.

1 g of 8-methyl-5 isoquinoline-sulfonic acid was suspended in 10 ml thionyl chloride. After adding 0.1 ml DMF the solution was heated for 5 h using a reflux condenser. Solvent was removed under vacuum and oily residue was two times concentrated with dichloromethane. The solid residue was suspended in 10 ml dichloromethane, filtered and washed with dichloromethane which yielded 354 mg yellowish solid. The solid matter was suspended in 10 ml ice water and pH value was adjusted to pH 6-7 with saturated sodium bicarbonate solution. After extracting with 5 ml dichloromethane the organic phase was dried over magnesium sulphate and added dropwise to a solution of 352 mg homopiperazine in 5 ml dichloromethane at 0° C. After 1 h stirring at 0° C. and 3 h at ambient temperature the solution was washed two times with 10 ml water, dried over magnesium sulphate, and concentrated. The resulting oil was chromatographically purified and precipitated in a water-acetone mixture. This resulted in 240 mg of 1-(8-methyl-5 isoquinoline-sulfonyl)homopiperazine as solid matter.

### Example 2

### Preparation of 1-(1-chloro-8-methoxy-5 isoquinoline-sulfonyl)homopiperazine

1-(1-chloro-8-methoxy-5 isoquinoline-sulfonyl)homopiperazine was manufactured according to FIG. 1. 18.5 g 2-methoxybenzaldehyde and 14 g-aminoacetylaldehyde dimethyl acetal were dissolved in 180 ml toluene and 3 h heated using a water separator. Solvent was removed and residue was dissolved in 105 ml dry THF. 10.3 ml ethyl chloroformate were added dropwise at −10° C. 20.6 ml trimethy phosphite were added at ambient temperature. After stirring 20 h solvent was distilled off and residue was concentrated three times with 50 ml toluene. The oily residue was dissolved under argon atmosphere in 180 ml dry dichloromethane. 90 ml titanium tetrachloride were added carefully and solution was heated 24 h using a reflux condenser. The solution was poured on 700 g ice and 340 ml concentrated ammoniac solution. Precipitate was filtered and extracted with 1 liter dichloromethane. The extract and filtrate were combined and extracted three times with 1 N hydrochloric acid. Combined aqueous phases were washed with 100 ml dichloromethane and ph value was adjusted to 10 with concentrated ammoniac solution. Aqueous phase was extracted three times with 350 ml dichloromethane. Combined organic phases were dried over sodium sulfate and solvent was removed to result in 14.5 g 8-methoxy-isoquinoline which is a brown oil.

12 g of the oil were dissolved in 50 ml acetic acid and 12 ml 30% H₂O₂ solution were added dropwise. After stirring 3 h at 70° C. further 12 ml 30% H₂O₂ solution were added and further 9 h were stirred at 70° C. At ambient temperature 150 ml saturated sodium carbonate solution were added. Aqueous phase was extracted 3 times with 250 ml dichloromethane and combined organic phases were dried over magnesium sulphate. Solvent was removed and residue was chromatographically purified which yielded 8.5 g 8-methoxy-isoquinoline-N-oxide, which is a yellow solid.

3.8 g of 8-methoxy-isoquinoline-N-oxide were dissolved under argon atmosphere in 57 ml phosphoryl chloride and heated for 3 h using a reflux condenser. Solvent was distilled off under vacuum and residue was dissolved in cold saturated sodium carbonate solution. Aqueous phase was extracted 3 times with 100 ml dichloromethane and combined organic phases were washed with 50 ml water and subsequently dried over sodium sulphate. Solvent was removed and residue was chromatographically purified which yielded 1.1 g 1-chloro-8-methoxy-isoquinoline.

800 mg of 1-chloro-8-methoxy-isoquinoline were dissolved in 5 ml ice cold sulphuric acid with subsequent dropwise adding of 5 ml oleum with further cooling. After stirring 2 h at 100° C. the solution was poured on ice water and the precipitate was filtered, washed with cold water, and vacuum dried which resulted in 1 g of 1-chloro-8-methoxy-5-isoquinoline-sulfonic acid which is a solid.

1.35 g of 1-chloro-8-methoxy-5-isoquinoline-sulfonic acid were suspended in 10 ml thionyl chloride. After adding 0.1 ml DMF the solution was heated for 2 h using a reflux condenser and stirred over night. Solvent was removed under vacuum and oily residue was two times concentrated with dichloromethane. The residue was suspended in 10 ml dichloromethane, filtered and washed with dichloromethane. The resulting yellow product of 618 mg was suspended in 10 ml ice water and pH value was adjusted to pH 7 with saturated sodium bicarbonate solution. After extracting with 5 ml dichloromethane the organic phase was dried over magnesium sulphate and added dropwise to a solution of 508 mg homopiperazine in 5 ml dichloromethane at 0° C. After 2 h stirring at 0° C. and 3 h at ambient temperature the solution was washed with 20 ml water, dried over magnesium sulphate, and concentrated. The residue was chromatographically purified and resuspended in 50 ml of a 1:1 mixture of dichloromethane and acetone. After incubating over night at 4° C. the precipitate was filtered and vacuum dried. This resulted in 147 mg of 1-(1-chloro-8-methoxy-5 isoquinoline-sulfonyl)homopiperazine as a solid.

### Example 3

### Preparation of 1-(1-hydroxy-8-acetyl-5 isoquinoline-sulfonyl)homopiperazine and 1-(8-acetyl-5 isoquinoline-sulfonyl)homopiperazine

1-(1-hydroxy-8-acetyl-5 isoquinoline-sulfonyl)homopiperazine and 1-(8-acetyl-5 isoquinoline-sulfonyl)homopiperazine is manufactured according to the synthesis way shown in FIG. 3.

### Example 4

### Preparation of 1-(1-hydroxy-7-acetyl-5 isoquinoline-sulfonyl)homopiperazine and 1-(7-acetyl-5 isoquinoline-sulfonyl)homopiperazine

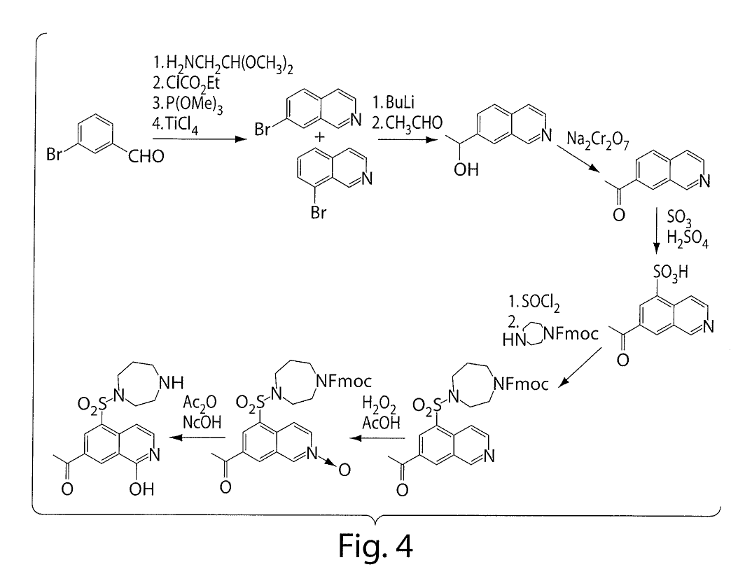

1-(1-hydroxy-7-acetyl-5 isoquinoline-sulfonyl)homopiperazine and 1-(7-acetyl-5 isoquinoline-sulfonyl)homopiperazine is manufactured according to the synthesis way shown in FIG. 4.

### Example 5

### Preparation of 1-(1-methyl-8-carboxamide-5 isoquinoline-sulfonyl)homopiperazine and 1-(1-ethyl-8-carboxamide-5 isoquinoline-sulfonyl)homopiperazine

1-(1-methyl-8-carboxamide-5 isoquinoline-sulfonyl)homopiperazine and 1-(1-ethyl-8-carboxamide-5 isoquinoline-sulfonyl)homopiperazine is manufactured according to the synthesis way shown in FIG. 5.

### Example 6

### Preparation of 1-(1-methyl-7-carboxamide-5 isoquinoline-sulfonyl)homopiperazine and 1-(1-ethyl-7-carboxamide-5 isoquinoline-sulfonyl)homopiperazine

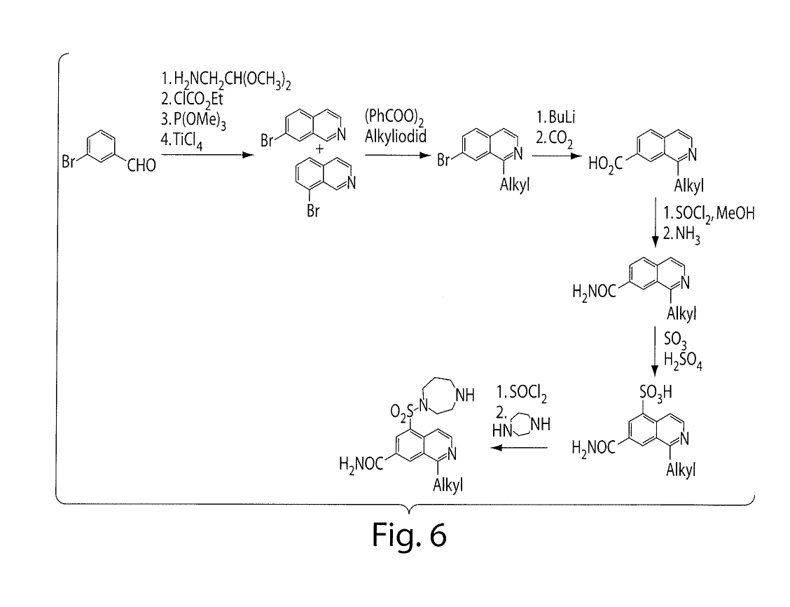

1-(1-methyl-7-carboxamide-5 isoquinoline-sulfonyl)homopiperazine and 1-(1-ethyl-7-carboxamide-5 isoquinoline-sulfonyl)homopiperazine is manufactured according to the synthesis way shown in FIG. 6.

### Example 7

### Preparation of 1-(8-aminoacetyl-5 isoquinoline-sulfonyl)homopiperazine

1-(8-aminoacetyl-5 isoquinoline-sulfonyl)homopiperazine is manufactured according to the synthesis way shown in FIG. 7.

### Example 8

### Preparation of 1-(6-aminoacetyl-5 isoquinoline-sulfonyl)homopiperazine

1-(6-aminoacetyl-5 isoquinoline-sulfonyl)homopiperazine is manufactured according to the synthesis way shown in FIG. 8.

### Example 9

### Preparation of 1-(7-aminoacetyl-5 isoquinoline-sulfonyl)homopiperazine

1-(7-aminoacetyl-5 isoquinoline-sulfonyl)homopiperazine is manufactured according to the synthesis way shown in FIG. 9.

### Example 10

### Preparation of 1-(8-aminomethyl-5 isoquinoline-sulfonyl) 2-methyl-piperazine

1-(8-aminomethyl-5 isoquinoline-sulfonyl) 2-methyl-piperazine is manufactured according to the synthesis way shown in FIG. 10.

### Example 11

### Preparation of 1-(1-methyl-8-trifluoromethyl-5 isoquinoline-sulfonyl) 2-methyl-piperazine

1-(1-methyl-8-trifluoromethyl-5 isoquinoline-sulfonyl) 2-methyl-piperazine is manufactured according to the synthesis way shown in FIG. 11.

### Example 12

### ROCK Inhibition with Fasudil Derivatives

The inhibitory effect of test compounds on rho kinase 2 (ROCK) was analyzed using recombinant active rho kinase 2 (Upstate, Lake Placid, N.Y., USA) and the a ROCK assay kit (Cell Biolabs Inc., San Diego, Calif., USA) following the manufacture's instruction. Recombinant rho kinase 2 was dissolved in kinase reaction buffer including the kinase substrate in the presence of the test compound. Test compounds were added in a final concentration of 0.1 to 100 μM. Assays without test compound served as control. Fasudil served as positive control for ROCK inhibition. Assays were incubated at 30° C. for 30-60 min and subsequently stopped by adding 50% of the reaction-volume of 0.5 M EDTA, pH 8.0. After washing steps phosphorylated kinase substrate was quantified using a specific anti-phospho-MYPT1 (Thr696) antibody and a HRP-conjugated secondary antibody.

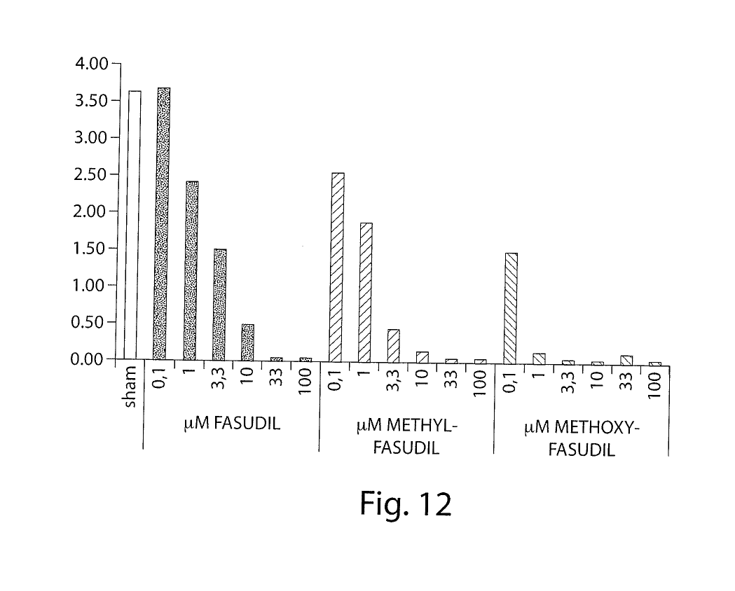

FIG. 12 shows the measured rho kinase 2 activity depending on the presence of Fasudil (positive control) or of the compounds 1-(8-methyl-5 isoquinoline-sulfonyl)homopiperazine (“methyl-fasudil”) or 1-(1-chloro-8-methoxy-5 isoquinoline-sulfonyl)homopiperazine (“methoxy-fasudil”). The tested compounds, particularly 1-(1-chloro-8-methoxy-5 isoquinoline-sulfonyl)homopiperazine, show an enhanced ROCK inhibition compared to Fasudil in corresponding concentration.

### Example 13

### Kinase Specificity Analysis

Based on a competition binding assay that quantitatively measures the ability of a test compound to compete with an immobilized, active-site directed ligand it is possible to scan the competitive effect of the test compound for a broad variety of kinases in parallel (KinomeScan, Ambit, San Diego, Calif., USA; Fabian et al., Nat. Biotechnol. 2005, 23, 329). Based on this analysis it is possible to assess the inhibitory specificity of a test compound. The Assay was performed with the compounds 1-(8-methyl-5 isoquinoline-sulfonyl)homopiperazine and 1-(1-chloro-8-methoxy-5 isoquinoline-sulfonyl)homopiperazine in comparison with the known ROCK inhibitor Fasudil each in 10 μM concentration. As result of the assay one obtains the percentage of competition of the active-site directed ligand for each of the over 400 kinases of the test due to the incubation with the test compounds. Competitions of more than 50% were regarded as significant indicating for inhibition of the particular kinase.

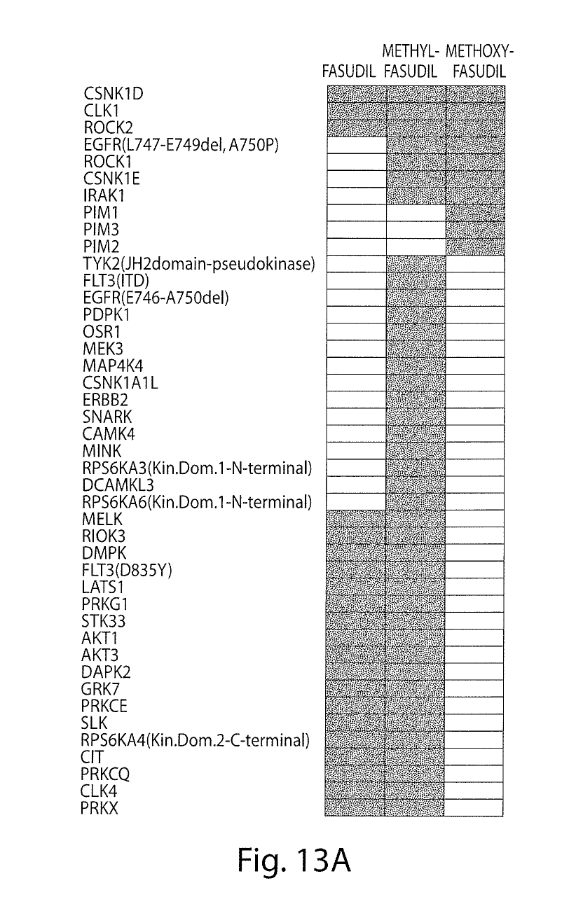

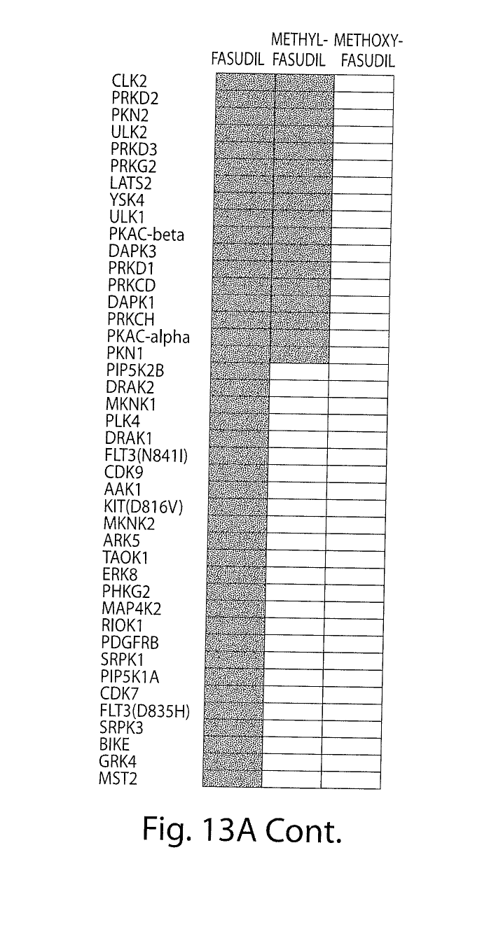

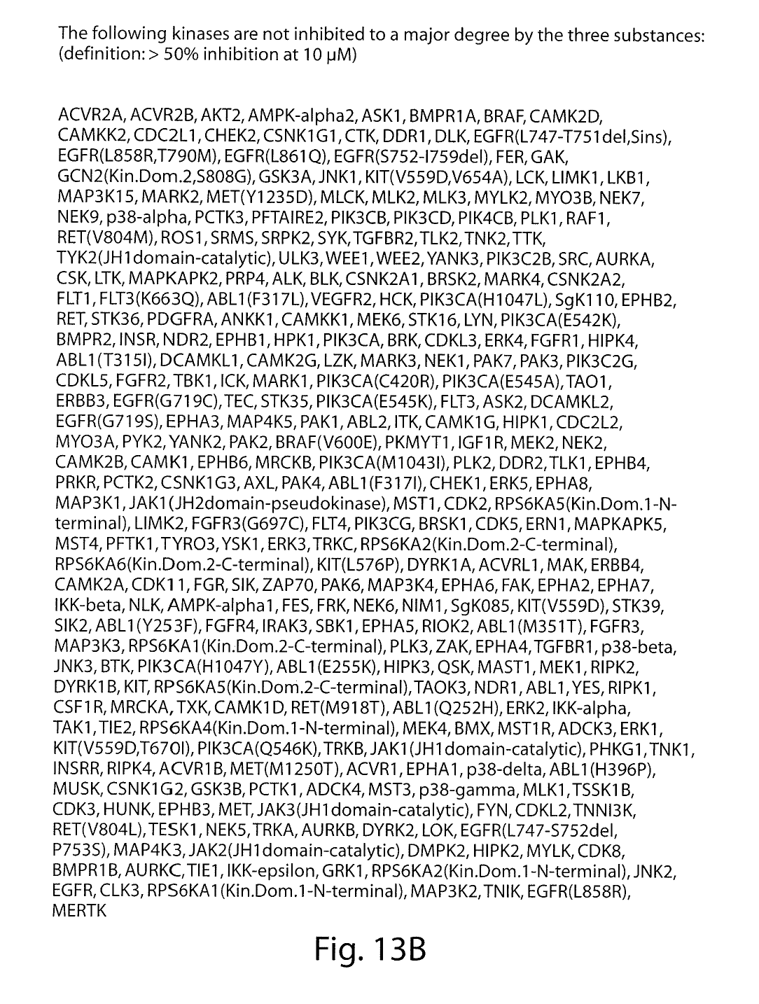

As shown in FIG. 13 A, 1-(1-chloro-8-methoxy-5 isoquinoline-sulfonyl)homopiperazine is a much more specific ROCK inhibitor than Fasudil. Compared to that, 1-(8-methyl-5 isoquinoline-sulfonyl)homopiperazine has a broader kinase interaction spectrum comparable to that of Fasudil but with a much stronger affinity to ROCK2 than Fasudil. This is equivalent with a considerably higher inhibitory activity for this type of kinase. FIG. 13 B lists the kinases which are inhibited neither by Fasudil nor by 1-(8-methyl-5 isoquinoline-sulfonyl)homopiperazine and 1-(1-chloro-8-methoxy-5 isoquinoline-sulfonyl)homopiperazine, whereas competition of more than 50% in this analysis is regarded as inhibitory.

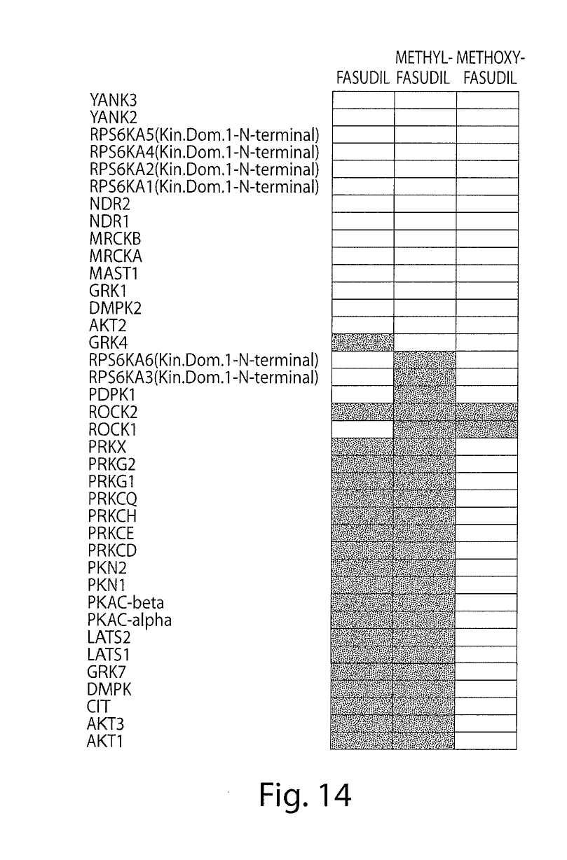

This assay reveals 1-(1-chloro-8-methoxy-5 isoquinoline-sulfonyl)homopiperazine as a very specific ROCK inhibitor that is expected to exhibit reduced side effects. Beside its ROCK inhibition, 1-(1-chloro-8-methoxy-5 isoquinoline-sulfonyl)homopiperazine exhibits mainly inhibitory effect only for PIM kinases and IRAK1. FIG. 14 shows the comparison of kinase active-site binding for Fasudil, 1-(8-methyl-5 isoquinoline-sulfonyl)homopiperazine and 1-(1-chloro-8-methoxy-5 isoquinoline-sulfonyl)homopiperazine with a focus on the ROCK related kinase. The hierarchical clustering represents a measure for the relationship between the various kinases.

### Example 14

### Neurite Outgrowth Analysis

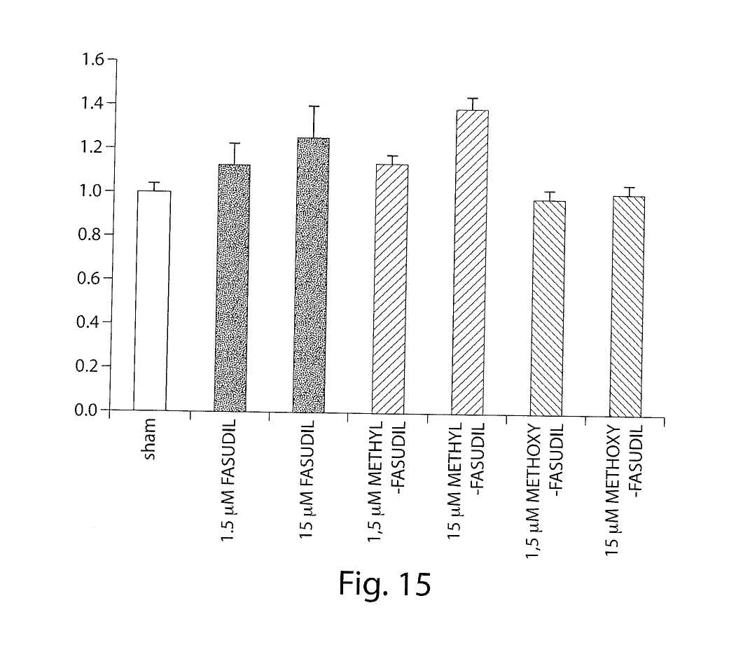

The effect of test compounds on neurite outgrowth can be assessed in vitro. Hippocampal primary neurons prepared from embryonic (E18) rats were cultured in Neurobasal medium (Invitrogen) enriched with B27, bFGF, Penicillin/Streptomycin, L-Glutamin. For the neurite outgrowth assay the medium is mixed with conditioned medium in a 1:1 ratio. The neurites were immunocytochemically stained using an antibody against the axonal marker neurofilament-light. Photographs were acquired at 40-fold magnification (Olympus IX81) and assembled to allow analysis of whole individual neurons. The test compounds 1-(8-methyl-5 isoquinoline-sulfonyl)homopiperazine and 1-(1-chloro-8-methoxy-5 isoquinoline-sulfonyl)homopiperazine were added to the medium in the final concentration of 1.5 μM or 15 μM 1 h after plating in comparison to adding water which served as negative control. Fasudil in the corresponding concentrations served as a positive control. After 2 days in culture the cells were fixed. Photographs and neurite tracing were performed in a blinded manner. FIG. 15 shows the neurite outgrowth promoting activity of 1-(8-methyl-5 isoquinoline-sulfonyl)homopiperazine (“methyl-fasudil”) in comparison to the negative control and in comparison to Fasudil. 1-(8-methyl-5 isoquinoline-sulfonyl)homopiperazine exhibit a superior neurite outgrowth promoting activity. 1-(1-chloro-8-methoxy-5 isoquinoline-sulfonyl) (“methoxy-fasudil”) homopiperazine did not significantly promote neurite outgrowth.

### Example 15

### In Vitro LTP Analysis

Long-term potentiation (LTP) is an in vitro model for the assessment of memory function. Therefore, it allows analysis of test compounds, e.g. the compounds of the invention, e.g. 1-(8-methyl-5 isoquinoline-sulfonyl)homopiperazine and 1-(1-chloro-8-methoxy-5 isoquinoline-sulfonyl)homopiperazine, for memory enhancing potential. Experiments were done on hippocampal slices from 3-4 week-old Wistar rats. The rats were sacrificed by decapitation without prior anesthesia. Brains were quickly removed and soaked in ice cold artificial cerebrospinal fluid (ACSF) containing: NaCl (124 mM), KCl (5 mM), Na2HPO4 (1.2 mM), NaHCO3 (26 mM), CaCl2 (2 mM), MgSO4 (2 mM), and glucose (10 mM), that was continuously bubbled with carbogen (95% O2, 5% CO2). Slices were then cut at 400 μm thickness using a vibratome and incubated in ACSF at room temperature for at least 1 h before starting recordings. All compounds used were diluted in ACSF at the concentrations needed and were prepared fresh on the day of recording from 100 mM stock solutions. To assure proper solubility of the compounds, stock solutions were made with DMSO. For recording, slices were transferred to a 4-channel slice chamber (Synchroslice, Lohmann Research Equipment) that allows simultaneous recording of 4 brain slices. Each slice was placed in a separate submerged type slice chamber were it was continuously superfused with temperature controlled (34° C.) ACSF or ACSF at a rate of 2 ml/min. Under visual control by a camera system, a bipolar stimulation electrode (Rhoades) was placed in the Schaffer collaterals and a single biphasic electrical stimulus of a duration of 200 μs and an amplitude of 200 μA was applied at 0.05 Hz. A platinum/tungsten electrode was then lowered into the CA1 dendritic layer under visual control until stable amplitudes of the recorded fEPSP were achieved. After a recording period of at least 10 min, the input-output relationship between stimulus amplitude and fEPSP amplitude was achieved separately for each slice. For recording, the stimulus amplitudes were chosen individually for each slice so that the resulting fEPSP showed 50% of the maximum amplitude from the IO curve. To induce LTP, 10 theta bursts were applied. Each burst consisted of 4 biphasic stimuli of 200 ms duration and 600 μA amplitude at a 10 ms interstimulus interval. The interburst interval was 200 ms. Each recording cycle started with a 15 min period in which electrical stimuli were applied at 0.05 Hz to assure stability of the fEPSP amplitude. Then, test the compound was washed in for a period of 30 min during which stimulation was continued at 0.05 Hz and fEPSPs were continuously recorded. LTP induction by theta burst stimulation was started 30 min after wash in. Recording was continued after LTP induction for at least 60 min, 30 min after LTP induction, compounds were washed out. All slices recorded simultaneously were treated with the same time schedule. From the recorded data, the amplitudes of the evoked fEPSP were automatically calculated by the recording software (Synchroslice data acquisition and analysis, LRE) as the negative peak of the postsynaptic signal with respect to baseline and plotted online. All recorded signals were digitally stored for later offline analysis, in particular for fEPSP negative slope calculation. From the stored single sweeps, the slope was calculated between 30% and 70% of the maximum fEPSP amplitude. To allow comparison of data obtained from different slices, fEPSP slopes were normalized to the control value (100%).

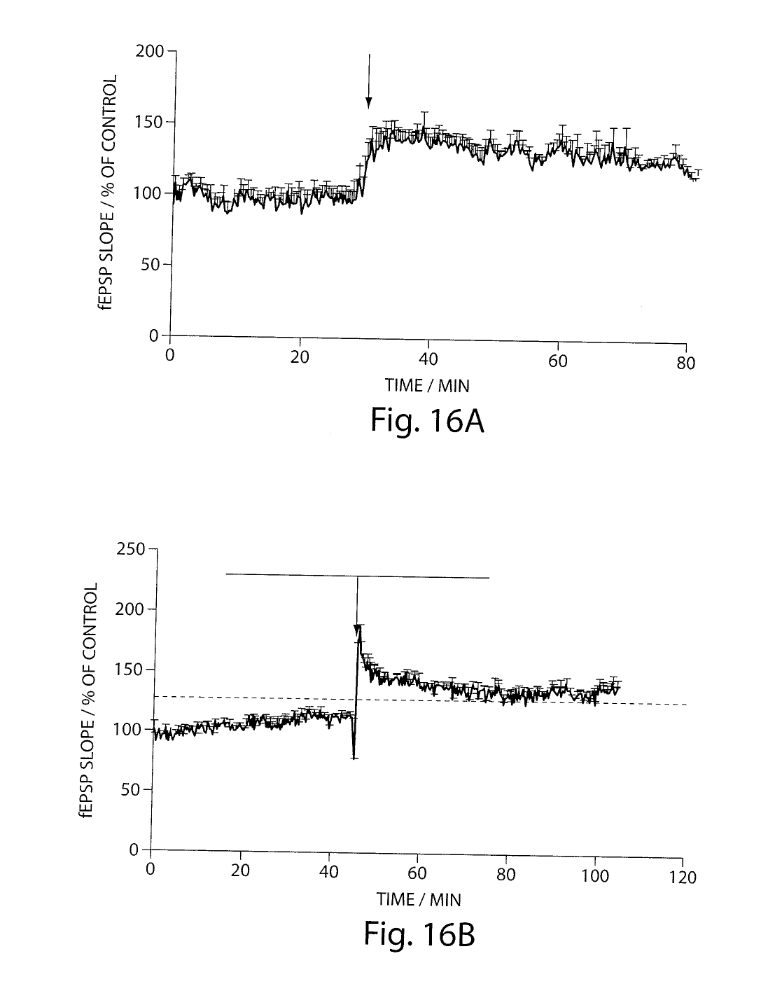

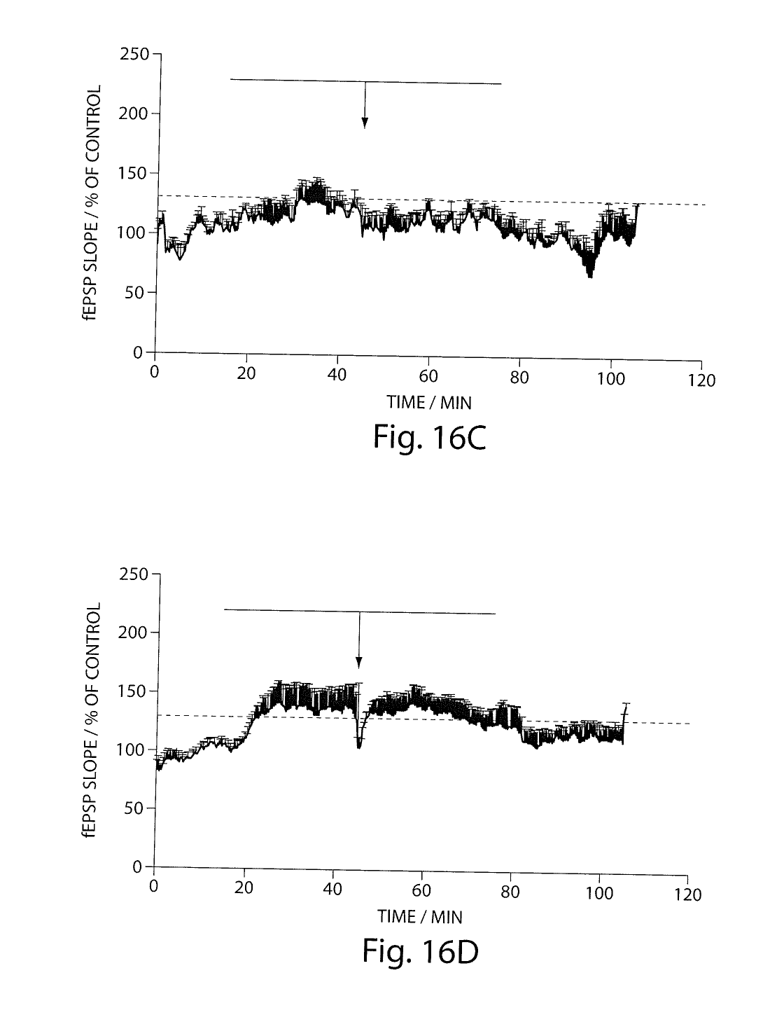

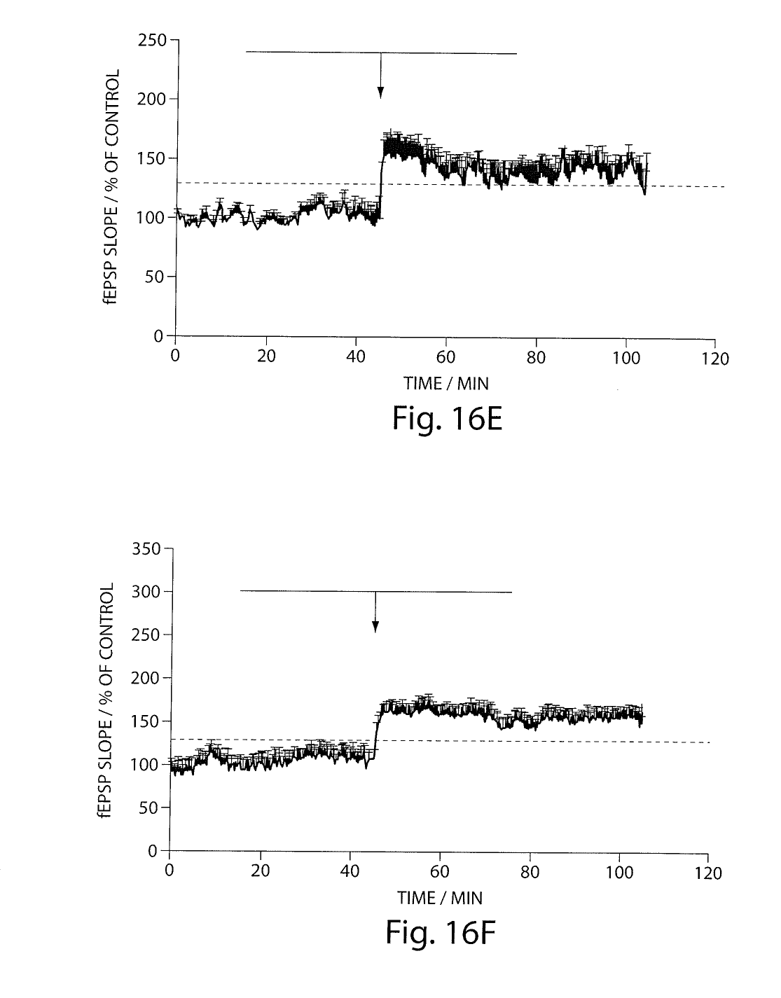

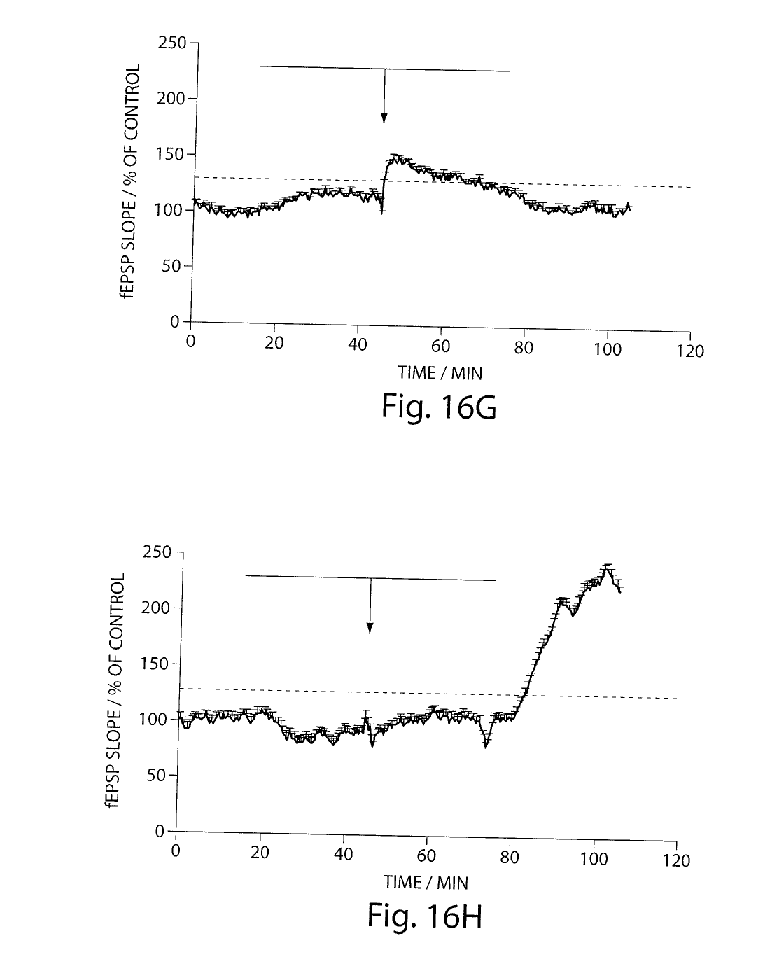

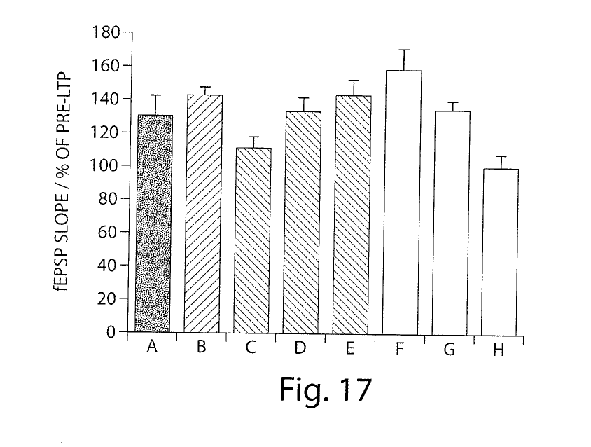

Effects induced by applied substances were tested for statistical significance using either the Student's t-test or the Mann-Whitney Rank Sum Test, significance was assumed if p<0.05. Measurements for each experimental condition were repeated six times. Results are given as means from n=5 slices and standard deviation (SD). The effects of 1-(8-methyl-5 isoquinoline-sulfonyl)homopiperazine and 1-(1-chloro-8-methoxy-5 isoquinoline-sulfonyl)homopiperazine were analyzed in comparison to sham incubated slices (as negative control) and fasudil incubated slices (as positive control). 1-(8-methyl-5 isoquinoline-sulfonyl)homopiperazine and 1-(1-chloro-8-methoxy-5 isoquinoline-sulfonyl)homopiperazine were tested in three concentrations (1 μM, 10 μM and 100 μM) and compared to 10 μM Fasudil. The results of the recording are shown in FIG. 16 (A. negative control; B: 10 μM fasudil; C: 1 μM 1-(8-methyl-5 isoquinoline-sulfonyl)homopiperazine; D: 10 μm 1-(8-methyl-5 isoquinoline-sulfonyl)homopiperazine; E: 100 μM 1-(8-methyl-5 isoquinoline-sulfonyl)homopiperazine; F: 1 μM 1-(1-chloro-8-methoxy-5 isoquinoline-sulfonyl)homopiperazine); G: 10 μM 1-(1-chloro-8-methoxy-5 isoquinoline-sulfonyl)homopiperazine); H: 100 μM 1-(1-chloro-8-methoxy-5 isoquinoline-sulfonyl)homopiperazine). 1-(1-chloro-8-methoxy-5 isoquinoline-sulfonyl)homopiperazine at 1 μM shows a clearly superior LTP stimulating effect compared to fasudil (FIG. 17). At 10 μM concentration the LTP induction is unaltered but the maintenance seems to be impaired. This could be due to the activation of undesired kinases and/or pathways at this concentration. At 100 μM, LTP induction is blocked completely. Upon removal of the substance there is a clear rebound of synaptic activity. One can hypothesize, that the LTP mechanism itself seems to be induced as in the low concentration but by an unknown secondary effect, due to the high concentration, the LTP is masked. Upon removal, the membrane potentials rapidly change which leads to the observed compensatory effect. 1-(8-methyl-5 isoquinoline-sulfonyl)homopiperazine at 1 μM concentration blocks LTP but in the highest concentration (100 μM) the ability of LTP induction is re-established. 1-(1-chloro-8-methoxy-5 isoquinoline-sulfonyl)homopiperazine shows significant enhancement of LTP and therefore is a superior candidate for memory enhancing.

### Example 16

### In Vivo Memory Assessment

Rats are one of the standard test systems for preclinical evaluation of age related cognitive impairments. Continuous subcutaneous administration of test compounds via osmotic mini pumps guarantees a stable plasma concentration and is therefore best for chronic application. In order to have a paradigm that investigates age-related memory impairment, 17 month old rats are used. Alternatively transgenic dementia-modelling animals (e.g. Alzheimers disease) are used. Animals are assigned to groups according to their treatment, one group only receives vehicle as control. Group sizes between 15 and 20 animals provide a proper statistical power depending on the number of groups investigated. For comparison of two groups, t-test statistics are used, for comparison of more than 2 groups, ANOVA corrected for multiple testing is applied. P-values of 0.05 are regarded as statistically significant. Experiments are performed in a blinded manner, including computer-generated probe randomizations and probe labeling, blindness of all experimenters to treatment identities until the end of the experiment, and separation of data analysis from experiment conduction. Animals are allowed to acclimate 1 week before starting the tests. Special care is taken to allow adequate access to food and water during trial, as well as for light-dark periods. One day before starting the tests osmotic mini pumps containing the test compounds or vehicle are implanted. The compounds according to the invention are tested for their memory enhancing ability in vivo by assays. Particularly suitable in vivo assays are described below in detail.

Radial arm maze: One day after surgery rats are habituated for 4 days in the radial arm maze. After the habituation phase animals are tested in the radial arm maze for 14 days using four randomly baited arms with a small food pellet and four non baited arms. Running in a non baited arm was counted as a reference memory error, re-entry in the same arm was counted as a working memory error as well as re-entry of a previous visited baited arm. The run was over when all baited arms were entered or the time limit of 480 s was reached.

Sacktor-disk: The test starts with a habituation trial, in which the animal is exposed to the apparatus for 10 min without shock. This is followed by successive training trials, in which the animal receives an electric shock every time the animal runs into the shock zone. Training consists of eight 10 min training trials, separated by 10 min rest intervals in their home cage. The animals are then tested 24 h later in a single probe trial. The probe trial measures the retention of long term stored spatial information by the increase in time between the placement of the animal into the apparatus and the initial entry into the shock zone. In addition, the retention of both short term and long term stored information is tested by the decrease in time spent in the shock zone (which is expressed rapidly after a single training session).

Morris Water maze (MWM): On day one a visible platform test is first performed. Extramaze cues are hidden by curtains and a platform is placed with a visible mark in the first quadrant of the MWM. The animal is placed at the opposite quadrant and swims until it finds the platform with a maximal time of 60 s. If it reaches the platform it is removed from the water, allowed to rest in its cage 30 s between each trial. Four trials are executed with the visible platform located in each of the 4 quadrants. This provides parameters about the sensorimotor and motivational features of the animals, the latency to reach the platform, the velocity and the distance moved to reach the platform.

On day two the animal is trained. In the pool with extramaze cues visible, it is placed at one of 4 randomly ordered start positions near the wall. The animal is supposed to swim to the submerged platform in a fixed position. If it fails to find the platform within 60 s, it is placed on the platform for 60 s. If it finds the platform within 60 s, it is allowed to stay there 60 s. The start location changes after each trial. The animal is trained to find the hidden platform with at least four trials per day. The animal is trained over as many days as it takes to reach the platform within 15 s. This provides parameters about the ability of learning and motor performance, escape latency, swim speed and swim distance. After the training sessions the probe trial is done. The platform is removed, the animal is placed into the pool at the opposite quadrant than the platform was formally located and the animal swims 60 s and is removed from the pool. This provide parameters on the percentage of time in quadrants of the MWM, number of crossings of the supposed platform positions, swim time, swim path length, swimming parallel to the wall, number of wall contacts and swimming speed.

### Example 17

### Testing Efficacy of PIM Inhibition for Cancer Treatment

PIM kinase activities can be assessed in vitro using standard methods in the art. In vitro assays are for example commercially available (e.g. HTScan® Pim-1 Kinase Assay Kit #7573 from Cellsignal, or the Pim-1 Kinase Assay/Inhibitor Screening Kit from Abnova). Typically, plates are coated with a protein or peptide substrate corresponding to targets of the protein kinase, which contains threonine residues that can be efficiently phosphorylated by Pim-1. The detector antibody specifically detects only the phosphorylated form of threonine, and is detected by color reactions. Alternatively, radiological methods can be used for example by using ATP with ³²P at the gamma position, and phosphorylation of target peptides can be monitored by exposure to sensitive screens (e.g. Fuji phosphoimager). Specificity of kinase inhibition can be assayed by screens against a large number of kinases in binding assays (e.g. Fabian, M. A. et al. A small molecule-kinase interaction map for clinical kinase inhibitors. Nat. Biotechnol. 23, 329-336 (2005); Karaman, M. W. et al. A quantitative analysis of kinase inhibitor selectivity. Nat. Biotechnol. 26, 127-132 (2008), which are hereby incorporated by reference). Efficacy of PIM inhibitors in cell culture can be tested as effects on proliferation of immortal cancer cell lines, e.g. the HeLa cell line and many others. Proliferation can be measured by counting cell density over time under a microscope, or by a number of biochemical assays. Invasion and spreading of cells can be assessed in soft agar tests, again using a large number of cancer cell lines. Induction of apoptosis in said cancer cells can be tested by assaying caspase 3 expression (e.g. by the Promega Caspase-glow assay). In vivo, anti cancer activity can be tested by transplanting cancer cell lines to animals, e.g. after immunosuppression, and monitoring the growth of those tumours for example by a reporter gene such as luciferase, and bioimaging using the single photon imaging boxes, or radiological assays like magnetic resonance imaging (MRI) or Micro-CT. Volume of the tumours also can be assessed post mortem in those animals. Typically the experiment is done in two groups of animals, one receiving placebo, and one receiving the drug. Sample sizes are typically 20 per group. In those animals also the lifespan and mortality can be monitored, and provide a clinically meaningful endpoint. Typically, a broad range of concentrations and application modes is tested in vivo to find an optimal dose range.

1.-15. (canceled)

16. A compound of Formula I:
wherein
R¹ is halogen;
R² is a member selected from the group consisting of halogen, —C(0)-R⁴, C₁₋₆ alkoxy, C₁₋₆ haloalkyl, —C(0)N(R⁴)R⁴, —N(R⁴)—C(0)-R⁴, —N(R⁴)R⁴, and —C(0)OR⁴;
R³ is a member selected from the group consisting of hydrogen, and C.1-6 alkyl;
each R⁴ is independently a member selected from the group consisting of hydrogen, C₁₋₆ alkyl and C₃₋₈ cycloalkyl; and
n is 0, 1, or 2.

17. The compound according to to claim 16
wherein
R¹ is halogen;
R² is a member selected from the group consisting of C₁₋₃ alkoxy, —C(0)-R⁴, —C(O) N(R⁴)R⁴, —N(R⁴)—C(0)-R⁴, —N(R⁴)R⁴, and —C(0)OR⁴,
R³ is a member selected from the group consisting of hydrogen, and C₁₋₃ alkyl;
each R⁴ is independently a member selected from the group consisting of hydrogen, C₁₋₃ alkyl and C₃₋₈ cycloalkyl; and n is 0, 1, or 2.

18. The compound of claim 16, wherein R² is C₁₋₆ alkoxy.

19. The compound of claim 16 that is selected from the group consisting of:

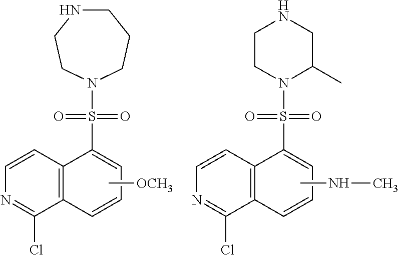

20. The compound 1-(1-chloro-8-methoxy-5 isoquinoline-sulfonyl)homopiperazine.

21. A method for improving memory in a subject, the method comprising administering to a patient in need thereof, a therapeutically effective amount of a compound according to claim 16.

22. A method for treating rho kinase 1 and/or 2 related conditions in a subject, the method comprising administering to a patient in need thereof, a therapeutically effective amount of a compound according to claim 16.

23. A method for treating PIM kinase related conditions in a subject, the method comprising administering to a patient in need thereof, a therapeutically effective amount of a compound according to claim 16.

24. The method of claim 23, wherein said condition is selected from the group consisting of ALL, CLL, AML, or CML, Hodgkin-Lymphoma and Non-Hodgkin Lymphoma.

25. A method for treating IRAK1 kinase related conditions in a subject, the method comprising administering to a patient in need thereof, a therapeutically effective amount of a compound according to claim 16.

26. The method of claim 25, wherein said condition is selected from the group consisting infection, atherosclerosis, sepsis, auto-immune diseases and cancer.

27. A method for treating conditions related to a kinase selected of the group consisting of CSNK1E, CSNK1A1L, CSNK1D, MERTK, SLK, IRAKI, STK10, MAPK12, PHKG2, MAPK11, MET, AXL, STK32B, AURKC, CLK3, RPS6KA6, PDGFRB, KDR, CDK2 in a subject, the method comprising administering to a patient in need thereof, a therapeutically effective amount of a compound of the formula:

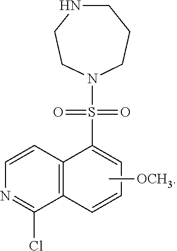

28. The method of claim 27, wherein the condition is selected from the group consisting of anxiety, depression, bipolar disorder, unipolar disorder, and post-traumatic stress disorder.

29. The method of claim 27 wherein the compound is 1-(1-chloro-8-methoxy-5 isoquinoline-sulfonyl)homopiperazine.

30. The method of claim 28 wherein the compound is 1-(1-chloro-8-methoxy-5 isoquinoline-sulfonyl)homopiperazine.
